(19) United States
(12) Patent Application Publication
(10) Pub. No.: US 2026/0191551 A1
(43) Pub. Date: Jul. 9, 2026

(54) SURGICAL INSTRUMENT

(71) Applicant: LIVSMED INC., Seongnam-si (KR)

(72) Inventors: Jae Hoon SHIN, Gwangju-si (KR); Jin Ah SIM, Yongin-si (KR); So Yeon KIM, Yongin-si (KR); Young Jae SONG, Seongnam-si (KR); Gyu Dong JEON, Seoul (KR); Yeon Taek JEONG, Suwon-si (KR)

(21) Appl. No.: 19/442,383

(22) Filed: Jan. 7, 2026

(30) Foreign Application Priority Data
Jan. 8, 2025 (KR) 10-2025-0002558

(51) Int. Cl. A61B 17/29; A61B 17/00; A61B 34/37

(52) U.S. Cl. CPC A61B 17/29; A61B 34/37; A61B 2017/00199; A61B 2017/00477; A61B 2017/2929; A61B 2017/2932

(57) ABSTRACT

A surgical instrument mountable on a surgical robot includes an end tool, a wire assembly, a connection part, and a driving part. The end tool includes one or more jaws and has at least one degree of rotational freedom. A first portion of the wire assembly is connected to the end tool. The wire assembly passes through an inside of the connection part. The end tool is coupled to a first side of the connection part, and the driving part is coupled to a second side of the connection part. The driving part includes a driving pulley assembly, a transition pulley assembly, and an auxiliary pulley assembly. The driving pulley assembly is rotatable and connected to a second portion of the wire assembly. The transition pulley assembly guides the wire assembly from the driving pulley assembly into the connection part. The auxiliary pulley assembly is adjacent to the transition pulley assembly and changes a path of the wire assembly between the transition and driving pulley assemblies.

### CROSS-REFERENCE TO RELATED APPLICATION

This application is based on and claims priority under 35 U.S.C. § 119 to Korean Patent Application No. 10-2025-0002558, filed on Jan. 8, 2025, in the Ministry of Intellectual Property (MOIP) of the Republic of Korea, the disclosure of which is incorporated by reference herein in its entirety.

### BACKGROUND

### 1. Field

The disclosure relates to a surgical instrument. More particularly, the disclosure relates to a surgical instrument that is mountable on a robot arm or manually operable for use in laparoscopic surgery or various other surgical procedures.

### 2. Description of the Related Art

Medically, surgery refers to the treatment of diseases by cutting, slitting, or manipulating the skin, mucous membranes, or other tissues using medical devices. In particular, open surgery, which involves making an incision in the skin at the surgical site to treat, reconstruct, or remove internal organs, may cause bleeding, side effects, pain, scarring, and the like. Recently, surgeries performed by making a small incision in the skin and inserting only medical devices, such as laparoscopes, surgical instruments, or microsurgical microscopes, or surgeries using robots have attracted attention as alternatives.

In this regard, a surgical robot refers to a robot that has the function of replacing surgical actions performed by a surgeon. Surgical robots have the advantage of performing operations with greater accuracy and precision than humans and of enabling remote surgery.

Surgical robots that are currently being developed worldwide may include a bone surgical robot, a laparoscopic surgical robot, a stereotactic surgical robot, and the like. In this case, the laparoscopic surgical robot is a robot that performs minimum invasive surgery using a laparoscope and small surgical instruments.

Moreover, a surgical robot generally includes a master robot and a slave robot. When a surgical operator manipulates a control lever (e.g., a handle) provided on the master robot, a surgical tool coupled to or grasped by a robot arm equipped on the slave robot may be manipulated to perform surgery.

Laparoscopic surgery is a cutting-edge surgical technique that involves making a small incision in the navel area to insert a laparoscope, which is an endoscope used to observe the inside of the abdomen, and the technique is expected to see significant development in the future. Today's laparoscopes are equipped with computer chips and have advanced to the point where they may provide magnified images that are clearer than those seen with the naked eye, and when used in conjunction with specially designed laparoscopic surgical instruments while viewing the surgical site on a monitor, a wide range of procedures may be performed.

The background art described above is technical information retained by the inventors in order to derive the disclosure or obtained by the inventors in the process of deriving the disclosure, and thus is not necessarily known art disclosed to the general public before the filing of the application.

### SUMMARY

The disclosure relates to a surgical instrument that is mountable on a robot arm or manually operable for use in laparoscopic surgery or various other surgical procedures, and provides a surgical instrument in which a path of a wire is optimized to reduce power loss of the wire.

According to an embodiment, a surgical instrument mountable on a robot arm includes an end tool including one or more jaws and having at least one degree of rotational freedom, a wire assembly having a first portion connected to the end tool, a connection part configured to extend in one direction, having an inside through which the wire assembly passes, and having a first side to which the end tool is coupled, and a driving part coupled to a second side of the connection part and configured to control a rotational motion of the end tool, wherein the driving part includes a driving pulley assembly configured to be rotatable around an axis and connected to a second portion of the wire assembly, a transition pulley assembly positioned such that at least a portion of the wire assembly is in contact therewith, and configured to guide the wire assembly to the inside of the connection part, the wire assembly extending from the driving pulley assembly, and an auxiliary pulley assembly positioned adjacent to the transition pulley assembly and configured to change a path of the wire assembly between the transition pulley assembly and the driving pulley assembly.

In an embodiment, a rotation shaft of the auxiliary pulley assembly may be positioned so as not to be parallel to a rotation shaft of the transition pulley assembly.

In an embodiment, a plane including a rotational surface of the transition pulley assembly and a plane including a rotational surface of the auxiliary pulley assembly may be perpendicular to each other.

In an embodiment, the rotation shaft of the auxiliary pulley assembly may be positioned parallel to a rotation shaft of the driving pulley assembly.

In an embodiment, the transition pulley assembly may include a first group of transition pulleys, a second group of transition pulleys, and a third group of transition pulleys, wherein the first group of transition pulleys, the second group of transition pulleys, and the third group of transition pulleys may each be configured to guide the wire assembly entering from a different direction.

In an embodiment, a rotation shaft of the first group of transition pulleys, a rotation shaft of the second group of transition pulleys, and a rotation shaft of the third group of transition pulleys may be positioned on a same plane.

In an embodiment, the rotation shaft of the first group of transition pulleys and the rotation shaft of the second group of transition pulleys may be positioned parallel to each other, and the rotation shaft of the third group of transition pulleys may be positioned perpendicular to the rotation shaft of the first group of transition pulleys and the rotation shaft of the second group of transition pulleys.

In an embodiment, the auxiliary pulley assembly may include a first group of auxiliary pulleys positioned adjacent to the first group of transition pulleys, and a second group of auxiliary pulleys positioned adjacent to the second group of transition pulleys.

In an embodiment, the transition pulley assembly may include a first transition pulley and a second transition pulley positioned to be rotatable around a same shaft, and the auxiliary pulley assembly may include a first auxiliary pulley and a second auxiliary pulley positioned to be rotatable around a same shaft.

In an embodiment, a diameter of the first transition pulley and a diameter of the second transition pulley may be different from each other, and a diameter of the first auxiliary pulley and a diameter of the second auxiliary pulley may be different from each other.

In an embodiment, a diameter of the first transition pulley and a diameter of the first auxiliary pulley may be equal to each other, and a diameter of the second transition pulley and a diameter of the second auxiliary pulley may be equal to each other.

In an embodiment, the auxiliary pulley assembly may be formed to be rotatable around a first shaft, and the transition pulley assembly may be formed to be rotatable around a second shaft that is not parallel to the first shaft.

In an embodiment, the wire assembly extending from the driving pulley assembly may enter the auxiliary pulley assembly in a first direction, pass through the auxiliary pulley assembly, and be redirected toward a second direction, then enter the transition pulley assembly in the second direction, pass through the transition pulley assembly, and be redirected toward a third direction that is not parallel to the first direction.

In an embodiment, each of the transition pulley assembly and the auxiliary pulley assembly may be formed as a pair to allow two strands of wire wound around the driving pulley assembly to be wound.

In an embodiment, the path of the wire assembly may include a straight path passing between the auxiliary pulley assembly and the transition pulley assembly, and a groove in which the wire assembly is wound around the auxiliary pulley assembly and a groove in which the wire assembly is wound around the transition pulley assembly may be positioned parallel to the straight path.

In an embodiment, the surgical instrument may further include a plate part on which the driving pulley assembly is positioned, and a wire guide assembly positioned on the plate part and on which the transition pulley assembly is positioned.

In an embodiment, the wire guide assembly may include a pulley accommodation space in which the transition pulley assembly is positioned, and a pulley shaft accommodation space in which a rotation shaft of the transition pulley assembly is accommodated.

According to an embodiment, a surgical instrument mountable on a robot arm includes an end tool including one or more jaws and having at least one degree of rotational freedom, a wire assembly having a first portion connected to the end tool, a connection part configured to extend in one direction, having an inside through which the wire assembly passes, and having a first side to which the end tool is coupled, and a driving part coupled to a second side of the connection part and configured to control a rotational motion of the end tool, wherein the driving part includes a driving pulley assembly configured to be rotatable around an axis and connected to a second side of the wire assembly, a transition pulley assembly positioned such that at least a portion of the wire assembly is in contact therewith, and configured to guide the wire assembly to the inside of the connection part, the wire assembly extending from the driving pulley assembly, and an auxiliary pulley assembly positioned adjacent to the transition pulley assembly and configured to change a path of the wire assembly between the transition pulley assembly and the connection part.

In an embodiment, the surgical instrument may further include a plate part on which the driving pulley assembly is positioned, wherein a distance from one surface of the plate part to the auxiliary pulley assembly may be shorter than a distance to the transition pulley assembly.

In an embodiment, an exit point of the transition pulley assembly may be positioned farther from a central axis of the connection part than an entry point of the auxiliary pulley assembly.

In an embodiment, a distance from an exit point of the auxiliary pulley assembly to a central axis of the connection part may be shorter than an inner radius length of the connection part.

In an embodiment, the auxiliary pulley assembly may include a plurality of auxiliary pulleys positioned to be rotatable around a same shaft, and the transition pulley assembly may include a plurality of transition pulleys positioned to be rotatable around a same shaft.

Other aspects, features, and advantages of the disclosure will become more apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a conceptual diagram of a surgical robot system including a surgical robot, according to an embodiment;

FIG. 2 is a perspective view of the surgical robot of FIG. 1;

FIG. 3 is an enlarged perspective view of some components of a first arm unit of FIG. 2;

FIG. 4 is a schematic perspective view of a surgical instrument according to an embodiment;

FIG. 5 is a perspective view of a driving part of the surgical instrument of FIG. 4;

FIG. 6 is a perspective view illustrating a state in which a cover is removed from the driving part of FIG. 5;

FIG. 7 is a perspective view of the driving part of FIG. 6, taken from a different angle;

FIG. 8 is a plan view of the driving part of FIG. 6;

FIG. 9 is a rear view of the driving part of FIG. 6;

FIG. 10 is a side view of the driving part of FIG. 6;

FIG. 11 is an exploded view illustrating a configuration of pulleys and wires of the driving part shown in FIG. 6;

FIG. 12 is an exploded view illustrating a configuration related to a first driving pulley among the configuration of pulleys and wires of the driving part shown in FIG. 6;

FIG. 13 is an exploded view illustrating a configuration related to a second driving pulley among the configuration of pulleys and wires of the driving part shown in FIG. 6;

FIG. 14 is an exploded view illustrating a configuration related to a third driving pulley among the configuration of pulleys and wires of the driving part shown in FIG. 6;

FIG. 15 is a perspective view of a wire guide assembly of the driving part of FIG. 6;

FIG. 16 is an exploded view illustrating a configuration of pulleys and wires according to a comparative example;

FIG. 17 is a diagram for explaining an arrangement state of transition pulleys and auxiliary pulleys shown in FIG. 7;

FIG. 18 is a diagram for explaining a modified example of the arrangement state of the auxiliary pulleys of FIG. 17;

FIG. 19 is a diagram of the transition pulley and the auxiliary pulleys shown in FIG. 17, taken from a different angle;

FIG. 20 is a diagram for explaining a modified example of an arrangement state of the transition pulleys of FIG. 19;

FIG. 21 is a perspective view of a driving part according to another embodiment;

FIG. 22 is an exploded view illustrating a configuration of pulleys of the driving part shown in FIG. 21;

FIG. 23 is a side view of the driving part of FIG. 21; and

FIG. 24 is a diagram for explaining an arrangement state of transition pulleys and auxiliary pulleys of the driving part of FIG. 21.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Because various transformations may be made to these embodiments, specific embodiments will be illustrated in the drawings and described in detail in the detailed description. Effects and features of the embodiments, and methods for achieving them will be clarified with reference to contents described below in detail with reference to the drawings. However, the embodiments may have different forms and should not be construed as being limited to the descriptions set forth herein.

In the description of the disclosure, when it is determined that specific explanations of known technologies may obscure the essence of the disclosure, the specific explanations are omitted.

In the following embodiments, singular forms are intended to include plural forms as well, unless the context clearly indicates otherwise. Although the terms “first”, “second”, and the like may be used to describe various components, such components should not be limited to the above terms. These terms are used only for the purpose of distinguishing one component from another.

In the following embodiments, it will be understood that the terms “comprise,” “include,” and “have” used herein specify the presence of stated features or components, but do not preclude the presence or addition of one or more other features or components.

In the following embodiments, it will be further understood that, when a unit, region, or component is referred to as being “on” another unit, region, or component, it may be directly or indirectly on the other unit, region, or component. That is, for example, intervening units, regions, or components may be present.

In the following embodiments, unless the terms “connect” and “couple” clearly mean otherwise in context, the terms not necessarily mean that two components are directly and/or fixedly connected to each other, and do not exclude the presence of another component between the two components.

Sizes of components in the drawings may be exaggerated or reduced for convenience of description. For example, because sizes and thicknesses of components in the drawings are arbitrarily illustrated for convenience of explanation, the following embodiments are not limited thereto.

In the following embodiments, the x-axis, the y-axis, and the z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

In cases where certain embodiments may be implemented otherwise, a specific process sequence may be performed differently from the described sequence. For example, two consecutively described processes may be performed substantially at the same time or performed in a sequence opposite to the described sequence.

A surgical robot 10 to which a motor pack according to an embodiment is applicable will be described first.

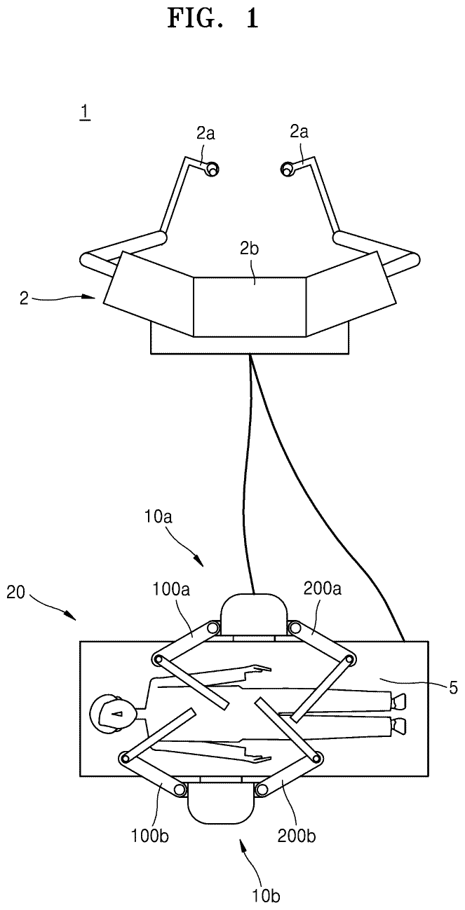

FIG. 1 is a conceptual diagram of a surgical robot system 1 including a surgical robot, according to an embodiment.

Referring to FIG. 1, the surgical robot system 1 may include a master robot 2 and the surgical robot 10.

The master robot 2 may include manipulating members and a display member, and the surgical robot 10 may include one or more robot arm units 100 and 200.

In detail, the master robot 2 may include manipulating members 2a such that a surgical operator is able to grip and manipulate the manipulating members 2a respectively with both hands. In addition, an image captured through a laparoscope is displayed as a screen image on a display member 2b of the master robot 2. Also, a virtual manipulation panel may be displayed independently or together with an image captured through a laparoscope or the like on the display member 2b. A detailed description of the arrangement, configuration, and the like of such a virtual manipulation panel will be omitted.

In some embodiments, the surgical robot 10 may include at least two robot arm units 100 and 200. In this case, the robot arm units 100 and 200 may be provided in a modular form that enables the robot arm units 100 and 200 to operate independently, and an algorithm for preventing collisions between the robot arm units 100 and 200 may be applied to the surgical robot system 1.

The surgical robot system 1 may include one or more surgical robots 10. FIG. 1 illustrates an embodiment in which the surgical robot system 1 includes two surgical robots 10a and 10b, and each of the surgical robots 10a and 10b includes two robot arm units 100 and 200, and a total of four robot arm units 100a, 200a, 100b, and 200b are positioned accordingly.

In an embodiment, surgical instruments SI may be attached to two or more of the robot arm units 100a, 200a, 100b, and 200b, and a laparoscope may be attached to at least one of the robot arm units 100a, 200a, 100b, and 200b. In addition, a surgical operator may use the master robot 2 to select any of the robot arm units 100a, 200a, 100b, and 200b for control. As described above, by directly controlling three or more surgical instruments using the master robot 2, the surgical operator may accurately and freely manipulate various instruments on a surgical bed 5 according to the surgical operator's intent, without requiring a surgical assistant.

Hereinafter, the configuration and operating principle of the surgical robot 10 will be described in detail.

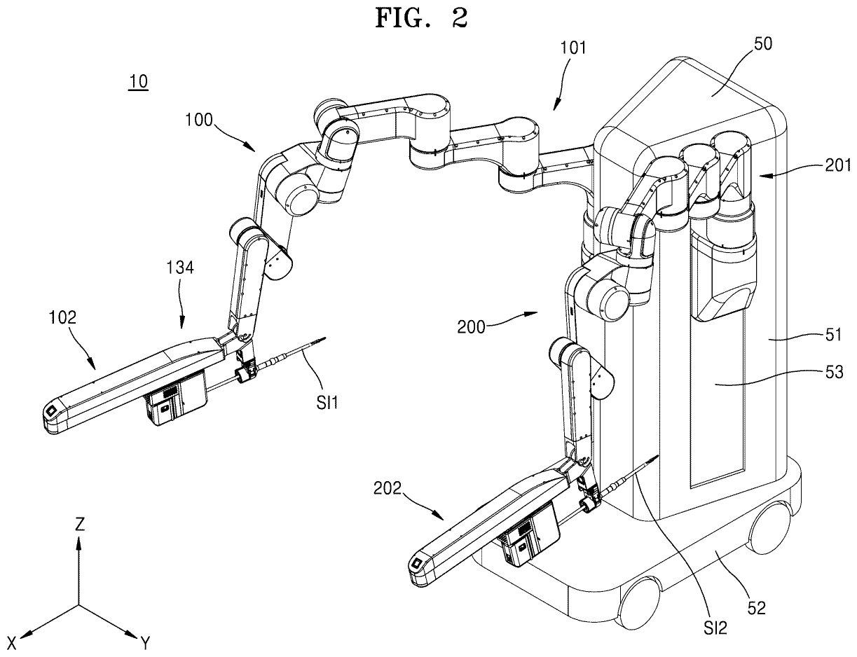

FIG. 2 is a perspective view of the surgical robot 10 of FIG. 1.

Referring to FIG. 2, the surgical robot 10 may include a body part 50, a first arm unit 100, and a second arm unit 200. FIG. 2 illustrates an embodiment in which the surgical robot 10 includes two robot arm units, and hereinafter, the robot arm units are defined as the first arm unit 100 and the second arm unit 200, respectively.

The body part 50 functions as a body of the surgical robot 10, on which the first arm unit 100 and the second arm unit 200 may be positioned. Also, the body part 50 may serve as a reference point for the driving of the first arm unit 100 and the second arm unit 200.

The body part 50 may include a first body 51 and a second body 52. The first arm unit 100 and the second arm unit 200 may be positioned on the first body 51, and the second body 52 may support the first body 51. Also, the second body 52 may include wheels, as shown in FIG. 2, thereby enabling the surgical robot 10 to move.

The body part 50 may have a lifting guide 53. The lifting guide 53 may be provided in correspondence with the number of robot arm units positioned on the body part 50. The lifting guide 53 may be recessed in one side of the body part 50, and each of the robot arm units 100 and 200 may be coupled to the lifting guide 53 so as to be slidable in a first direction.

In describing the disclosure, a portion closer to the body part 50 will be referred to as a proximal end, and a portion farther from the body part 50 will be referred to as a distal end. For example, a portion of the first arm unit 100 closer to the body part 50 is defined and described as a proximal end 101 of the first arm unit 100, and a portion of the first arm unit 100 farther from the body part 50 is defined and described as a distal end 102 of the first arm unit 100. Similarly, a portion of the second arm unit 200 closer to the body part 50 is defined and described as a proximal end 201 of the second arm unit 200, and a portion of the second arm unit 200 farther from the body part 50 is defined and described as a distal end 202 of the second arm unit 200.

The first arm unit 100 may be positioned on one side of the body part 50 and may have a first surgical instrument SI1 mounted thereon. The surgical robot 10 may drive the first arm unit 100 to adjust the position and posture of the first surgical instrument SI1.

A first arm connection part (not shown) may include a plurality of connection links, and a posture thereof may be determined according to the driving of each connection link. A remote center of motion (RCM) point RCM1 of the first surgical instrument SI1 may be determined according to the posture of the first arm connection part. In this case, the RCM point of the first surgical instrument SI1 refers to an imaginary center point that serves as a rotational reference for the first surgical instrument SI1. The first surgical instrument SI1 may perform a yaw motion and a pitch motion by rotating around the RCM point.

The second arm unit 200 may be positioned on another side of the body part 50 and may have a second surgical instrument SI2 mounted thereon. The surgical robot 10 may drive the second arm unit 200 to adjust the position and posture of the second surgical instrument SI2.

A second arm connection part (not shown) may include a plurality of connection links, and a posture thereof may be determined according to the driving of each connection link. An RCM point of the second surgical instrument SI2 may be determined according to the posture of the second arm connection part. In this case, the RCM point of the second surgical instrument SI2 refers to an imaginary center point that serves as a rotational reference for the second surgical instrument SI2. The second surgical instrument SI2 may perform a yaw motion and a pitch motion by rotating around the RCM point.

Each of the arm units may include a plurality of arm connection links and a plurality of arm extension links. The arm connection links and the arm extension links may rotate around respective reference axes thereof, and such rotational motions allow the arm unit to adjust the posture and position of the surgical instrument within an operational range.

In this case, a detailed description of the arm connection links and the arm extension links will be omitted.

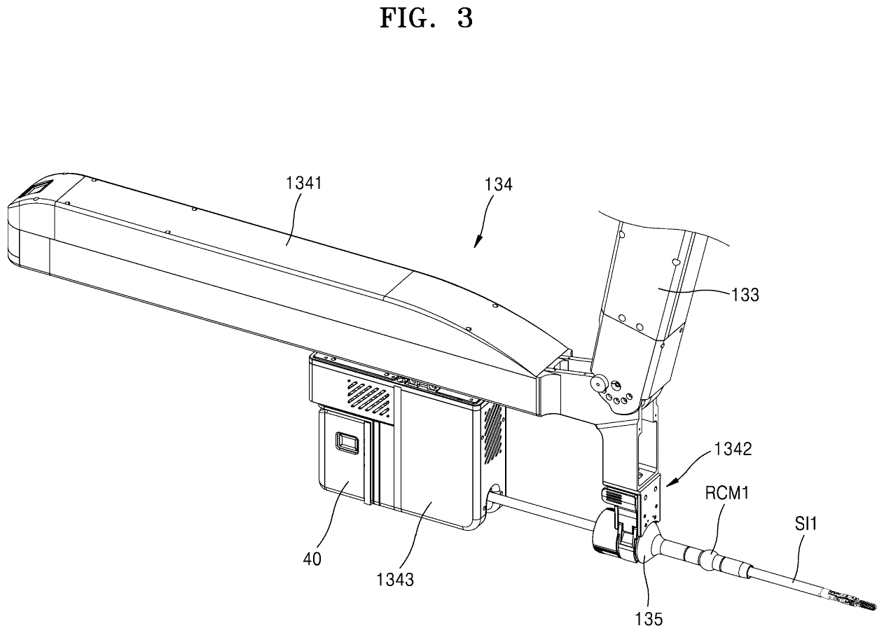

FIG. 3 is an enlarged perspective view of some components of the first arm unit 100 of FIG. 2.

Referring to FIGS. 2 and 3, the first arm unit 100 according to an embodiment may include a first arm slide link 134 on the side of the distal end 102.

In this case, the first arm slide link 134 may allow sliding movement of the first surgical instrument SI1.

The first arm slide link 134 may be coupled to one end, that is, on a distal end side, of a first arm third extension link 133, and the first surgical instrument SI1 may be positioned on the first arm slide link 134.

The first arm slide link 134 may include a translation arm 1341, a slide motor pack 1343, a driving part 40, and a trocar holder 1342.

The translation arm 1341 is coupled to one end of the first arm third extension link 133 and may move together with the first arm third extension link 133. That is, when the first arm third extension link 133 is driven, a posture of the translation arm 1341 may change accordingly.

The slide motor pack 1343 may provide a driving force for the sliding movement of the first surgical instrument SI1. The slide motor pack 1343 may include one or more first motors, as well as various components configured to generate and transmit a driving force.

The first surgical instrument SI1 is connected to the slide motor pack 1343 through the driving part 40 and may be linearly moved by the slide motor pack 1343.

Also, the driving part 40 may receive a driving force from the slide motor pack 1343 and transmit the driving force to an end tool of the first surgical instrument SI1.

That is, the slide motor pack 1343 receives power from an external source to generate a driving force, and the driving force generated by the slide motor pack 1343 may be transmitted to the first surgical instrument SI1, whereby the first surgical instrument SI1 is able to perform a pitch motion, a yaw motion, an actuation motion, and a roll motion.

The trocar holder 1342 is positioned in a region of the translation arm 1341, and a trocar 135 may be mounted on the trocar holder 1342. In detail, the trocar holder 1342 may be positioned near a distal end of the first arm third extension link 133.

The trocar 135 is mountable on the trocar holder 1342, and the first surgical instrument SI1 may be coupled to the trocar 135. The first surgical instrument SI1 may be mounted through the trocar 135. Also, the first surgical instrument SI1 may be partially supported by the trocar 135 and be slidable.

The RCM point RCM1 of the first surgical instrument SI1 may be defined on one side of the trocar 135. That is, the trocar 135 may provide the RCM point on one side, which serves as a reference point for rotational movements of the first surgical instrument SI1, including a yaw motion and a pitch motion. When the posture of the first arm connection part is determined, a position of the RCM point defined on the trocar 135 is also determined, and the position of the RCM point may remain fixed even when the first surgical instrument SI1 moves in a sliding manner.

Hereinafter, the surgical instrument according to an embodiment will be described in detail.

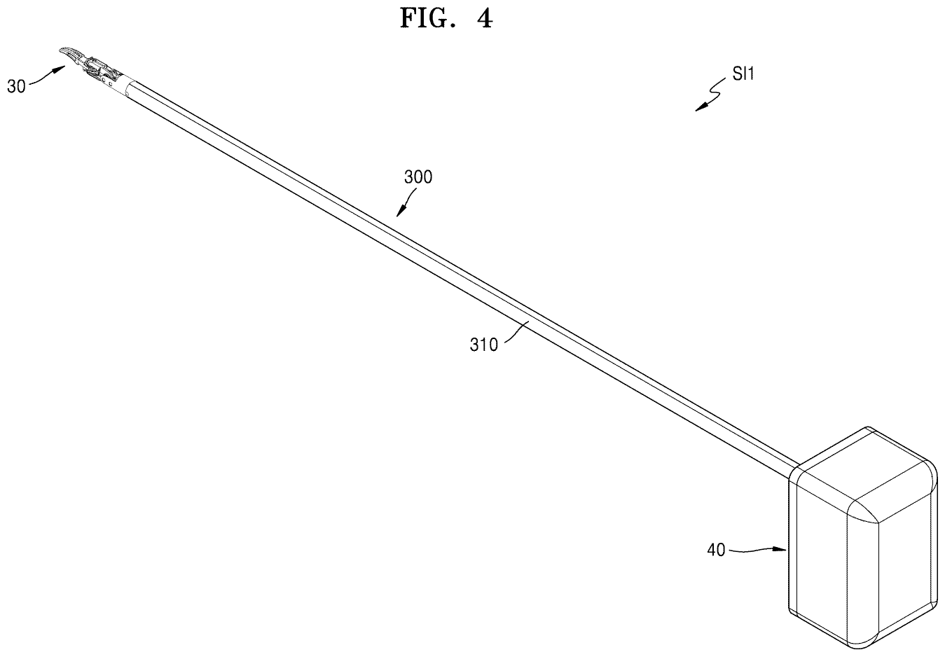

FIG. 4 is a schematic perspective view of a surgical instrument SI1 according to an embodiment.

The surgical instrument SI1 according to an embodiment may include an end tool 30, the driving part 40, and a power transmission part 300, and the power transmission part 300 may include a connection part 310.

In this case, the connection part 310 is formed in the shape of a hollow shaft, in which one or more wires (to be described below) may be accommodated, and may have one end to which the driving part 40 is coupled and another end to which the end tool 30 is coupled, thereby serving to connect the driving part 40 to the end tool 30.

The driving part 40 may be provided at one end of the connection part 310 and provide an interface capable of being coupled to the robot arm unit 100 or the like (see FIG. 2). Accordingly, when a user operates the master robot 2 (see FIG. 1), a motor (not shown) of the robot arm unit 100 or the like (see FIG. 2) may be activated such that the end tool 30 of the surgical instrument SI1 may perform a corresponding motion, and a driving force of the motor (not shown) may be transmitted to the end tool 30 via the driving part 40. In other words, the driving part 40 may be described as an interface that connects the surgical instrument SI1 to the surgical robot 10.

The end tool 30 may be provided at another end of the connection part 310 and perform necessary motions for surgery by being inserted into a surgical site. As an example of the end tool 30, a pair of jaws for performing a grip motion may be used. However, the concept of the disclosure is not limited thereto, and various devices for performing surgery may be used as the end tool 30. For example, a configuration such as a monopolar electrocautery may also be used as the end tool, and forceps, a needle holder, a dissector, a stapler, a clip applier, and the like may also be used as the end tool. Also, as the end tool, surgical tools such as monopolar dissectors, monopolar scissors, monopolar hooks, monopolar spatulas, bipolar dissectors, bipolar forceps, and vessel sealers may be used for electrocautery.

The aforementioned end tool 30 may be connected to the driving part 40 via the power transmission part 300 and receive a driving force of the driving part 40 through the power transmission part 300 to perform a motion necessary for surgery, such as a gripping motion, a cutting motion, a suturing motion, or the like.

In this case, the end tool 30 of the surgical instrument according to an embodiment is configured to be rotatable in at least two directions. For example, the end tool 30 may be configured to perform a pitch motion around one rotation shaft, while simultaneously performing a yaw motion and an actuation motion around another rotation shaft. The end tool 30 may also be capable of performing a roll rotational motion around the connection part (shaft) as a rotation shaft. In this case, the connection part 310 and the end tool 30 may perform a roll rotation together, or the end tool 30 alone may perform a roll rotation independently. In other words, the end tool may have at least one degree of rotational freedom.

Hereinafter, the driving part of the surgical instrument of FIG. 4 will be described in more detail.

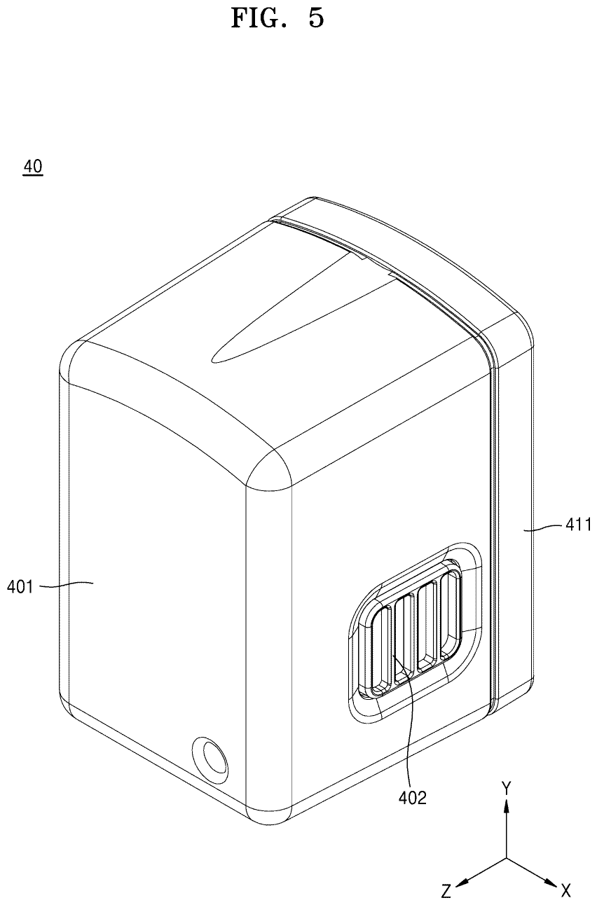

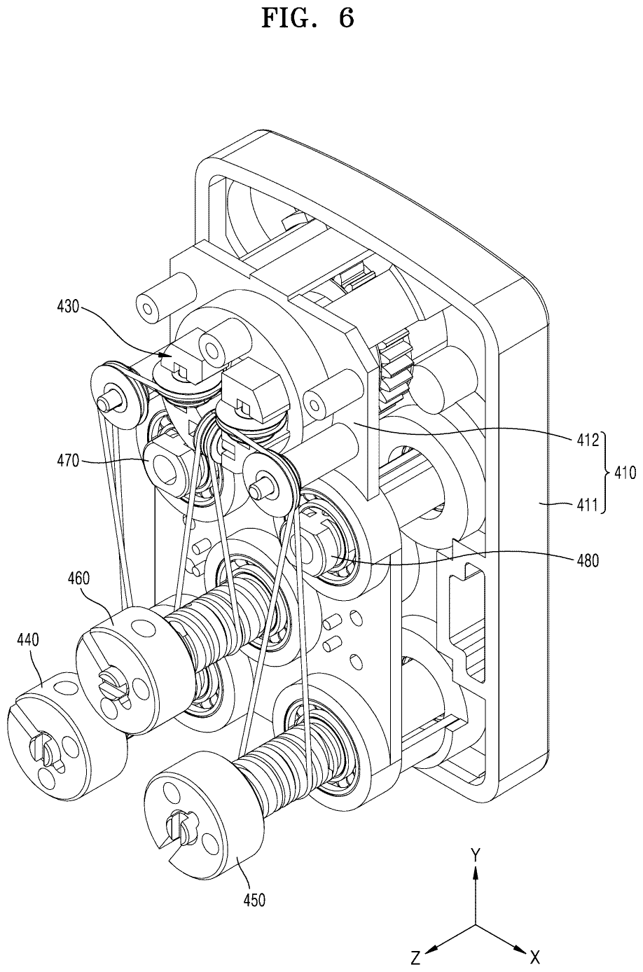

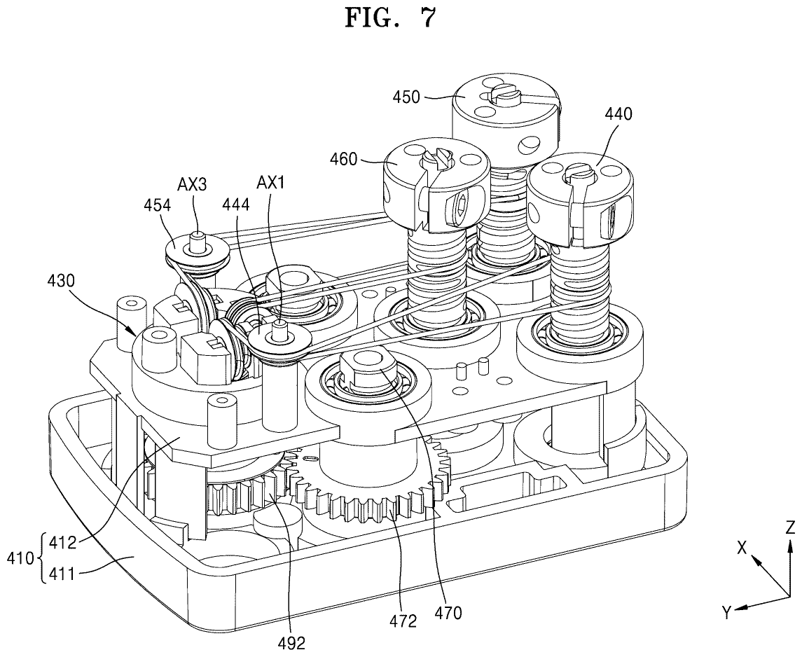

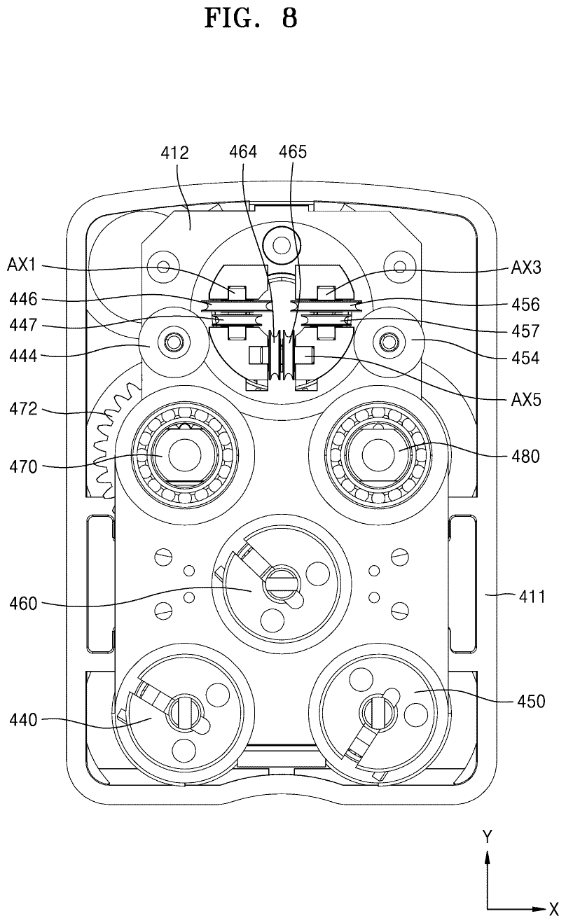

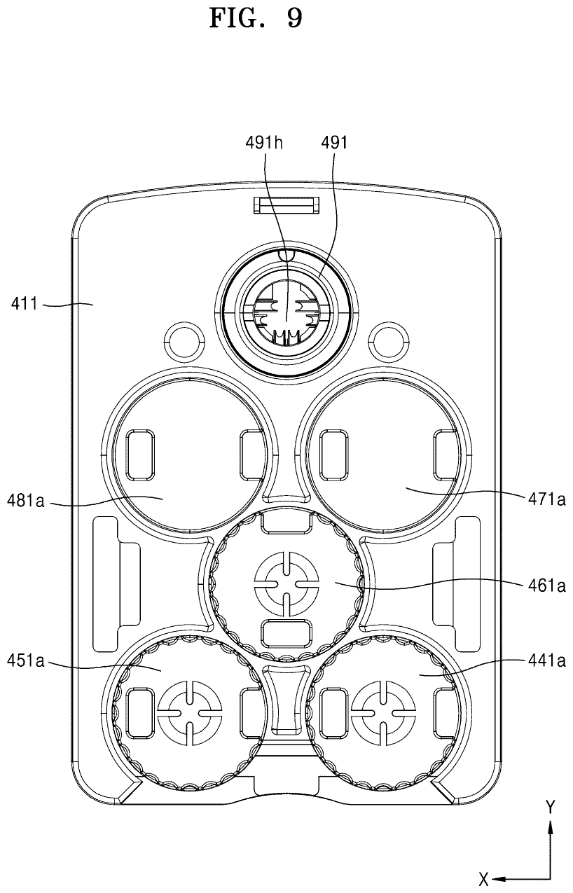

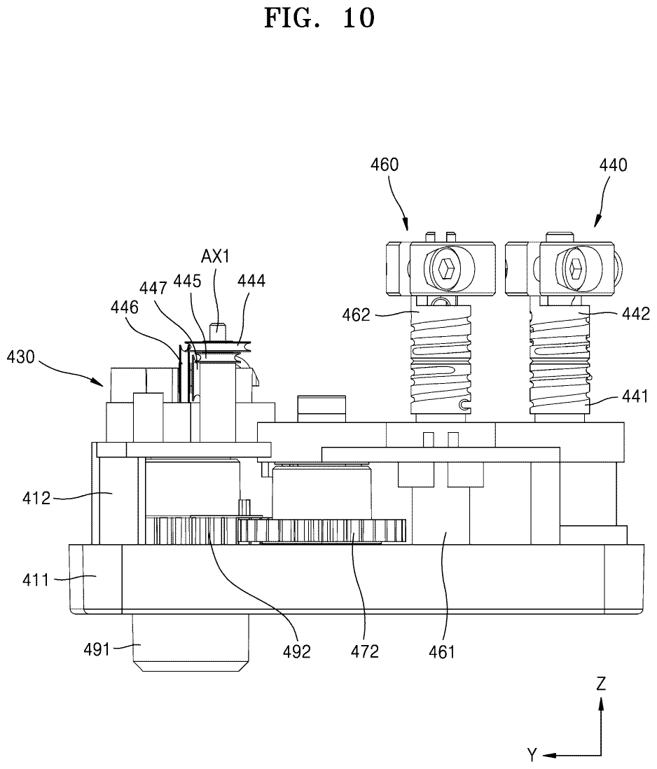

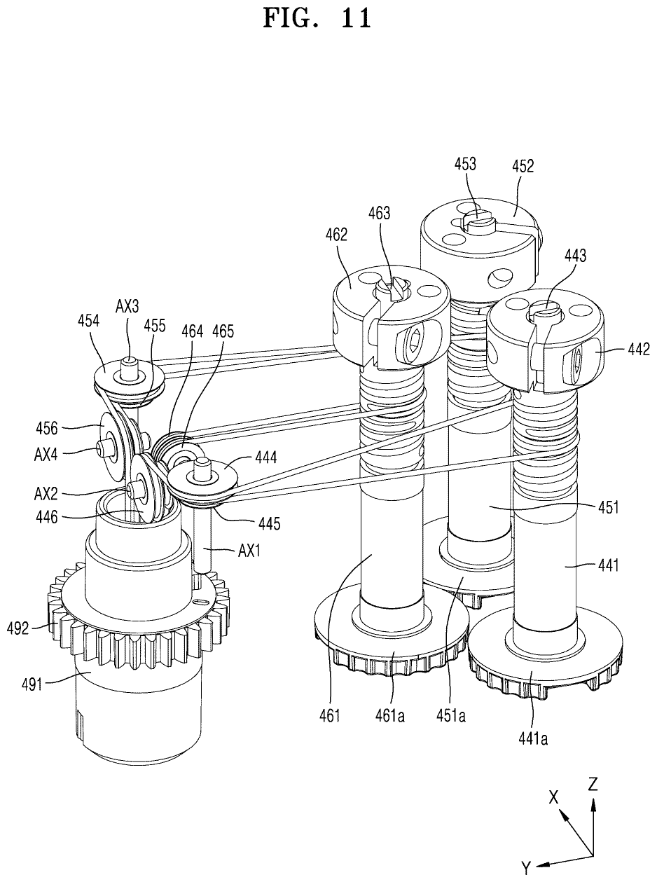

FIG. 5 is a perspective view of the driving part 40 of the surgical instrument of FIG. 4, and FIG. 6 is a perspective view illustrating a state in which a cover is removed from the driving part 40 of FIG. 5. FIG. 7 is a perspective view of the driving part of FIG. 6, taken from a different angle. FIG. 8 is a plan view of the driving part of FIG. 6, FIG. 9 is a rear view of the driving part of FIG. 6, and FIG. 10 is a side view of the driving part of FIG. 6. FIG. 11 is an exploded view illustrating a configuration of pulleys and wires of the driving part shown in FIG. 6.

Referring to FIGS. 5 to 11, the driving part 40 according to an embodiment may include a plate part 410, a cover 401, a wire guide assembly 430, a driving pulley assembly (reference numeral not shown), a transition pulley assembly (reference numeral not shown), an auxiliary pulley assembly (reference numeral not shown), and a wire assembly (reference numeral not shown).

The driving pulley assembly is a concept referring to a driving pulley itself or referring to a plurality of driving pulleys. That is, the driving pulley assembly may include a plurality of driving pulleys, and when describing common contents for each driving pulley, the driving pulley assembly or the driving pulley will be referred to for convenience of explanation.

Similarly, the wire assembly may include a plurality of wires, and when describing common contents for each wire, the wire assembly or the wire will be referred to for convenience of explanation.

Also, the auxiliary pulley assembly may include a plurality of auxiliary pulleys, and when describing common contents for each auxiliary pulley, the auxiliary pulley assembly or the auxiliary pulley will be referred to for convenience of explanation.

In addition, the transition pulley assembly may include a plurality of transition pulleys, and when describing common contents for each transition pulley, the transition pulley assembly or the transition pulley will be referred to for convenience of explanation.

The cover 401 is coupled to the plate part 410 and may protect pulleys and wires inside the driving part 40. An unlock button 402 may be provided on a side surface of the cover 401. The unlock button 402 may function to decouple the driving part 40 from the slide motor pack 1343. For example, when the driving part 40 is coupled to the slide motor pack 1343, pressing the unlock button 402 may cause a locking part (not shown) to be decoupled from a coupling part (not shown) of the slide motor pack 1343, such that the driving part 40 may be brought into a removable state from the slide motor pack 1343.

The plate part 410 may include a base plate 411 that provides a coupling surface for coupling to the aforementioned slide motor pack 1343, and a middle plate 412 that is positioned on a side opposite to the coupling surface of the base plate 411 and coupled to the base plate 411. That is, the base plate 411 and the middle plate 412 may be members that are separately provided and coupled to each other. However, the concept of the disclosure is not limited thereto, and the base plate 411 and the middle plate 412 may be integrally provided as a single body.

In this case, the connection part 310 having a shaft shape may be coupled to the coupling surface of the base plate 411. In detail, a shaft connector 491 may be positioned in the plate part 410, and the shaft connector 491 may connect the aforementioned connection part 310 to the driving part 40. In other words, the end tool 30 may be coupled to one end of the connection part 310, and the shaft connector 491 may be coupled to another end of the connection part 310, such that the connection part 310 may be coupled to the driving part 40 through the shaft connector 491.

In this case, the shaft connector 491 may serve as a passage through which wires passing through a through-hole of the wire guide assembly 430 to be described below pass. The wires may extend to the inside of the connection part 310 through a hollow 491h of the shaft connector 491.

In addition, motor coupling parts, to which motors (not shown) configured to drive driving pulleys are coupled, may be positioned on the coupling surface of the base plate 411.

In this regard, the motor coupling parts may be directly connected to the respective driving pulleys, or may be indirectly connected to the driving pulleys via gears.

Referring to FIG. 9, the driving part 40 according to an embodiment may include a first motor coupling part 441a, a second motor coupling part 451a, a third motor coupling part 461a, a fourth motor coupling part 471a, and a fifth motor coupling part 481a. In this case, the first motor coupling part 441a may function as a first jaw driving motor coupling part, the second motor coupling part 451a may function as a second jaw driving motor coupling part, the third motor coupling part 461a may function as a pitch driving motor coupling part, and the fourth motor coupling part 471a may function as a roll driving motor coupling part.

In this regard, each motor coupling part may be formed in the shape of a rotatable flat plate, and one or more coupling holes to which the motor (not shown) may be coupled may be formed therein.

The motors (not shown) provided in the slide motor pack 1343 may be coupled to the aforementioned motor coupling parts of the driving part 40, such that the driving part 40 is operated by the driving of the motors.

In some embodiments, the middle plate 412 may provide a region in which the driving pulley assembly is positioned. In addition, the middle plate 412 may provide a region in which the wire guide assembly 430 to be described below is positioned.

Referring to FIG. 6 or the like, the driving pulley assembly may include a first driving pulley 440, a second driving pulley 450, a third driving pulley 460, a fourth driving pulley 470, and a fifth driving pulley 480.

In this case, the first driving pulley 440 may be a pulley related to a rotational motion of a first jaw, and the second driving pulley 450 may be a pulley related to a rotational motion of a second jaw. Also, the third driving pulley 460 may be a pulley related to a pitch motion of the end tool 30, and the fourth driving pulley 470 may be a pulley related to a roll rotation of the connection part 310 and the end tool 30. This will be described in detail below.

Each of the driving pulleys may be formed as a single body or may be configured with a plurality of parts. For example, each of the driving pulleys may include a first part, a second part, and a driving pulley rotation shaft.

In detail, the first driving pulley 440 may include a first-1 part 441, a first-2 part 442, and a first driving pulley rotation shaft 443. In this regard, the first-1 part 441 and the first-2 part 442 are parts to which wires are respectively connected and wound, and may be coupled to the first driving pulley rotation shaft 443 to rotate together with the first driving pulley rotation shaft 443.

The first motor coupling part 441a may be formed at one end of the first-1 part 441. That is, the first motor coupling part 441a may be directly coupled to the first driving pulley 440, and when the first motor coupling part 441a, which is coupled to a first jaw driving motor (not shown), rotates, the first-1 part 441, the first driving pulley rotation shaft 443, and the first-2 part 442, which are directly coupled thereto, may rotate together. That is, when the first motor coupling part 441a rotates, the first driving pulley 440 may rotate.

A groove around which a wire is wound may be formed on the side of another end of the first-1 part 441. That is, the groove may be formed in a region of the first-1 part 441 adjacent to the first-2 part 442. Also, a groove around which a wire is wound may be formed in a region of the first-2 part 442 adjacent to the first-1 part 441. One end of the first-2 part 442 may be adjacent to the first-1 part 441, and a first driving pulley head 442a may be formed at another end of the first-2 part 442.

In addition, the wire wound around the first-1 part 441 and the wire wound around the first-2 part 442 may be wound in opposite directions to each other. For example, the wire wound around the first-1 part 441 may be wound in a clockwise direction, and the wire wound around the first-2 part 442 may be wound in a counterclockwise direction. Accordingly, when the first driving pulley 440 rotates in one direction, one wire may be wound around the first driving pulley 440, and another wire may be unwound from the first driving pulley 440.

Also, the second driving pulley 450 may include a second-1 part 451, a second-2 part 452, and a second driving pulley rotation shaft 453. In this case, the second-1 part 451 and the second-2 part 452 are portions to which wires are respectively connected and wound, and may be coupled to the second driving pulley rotation shaft 453 to rotate together with the second driving pulley rotation shaft 453.

The second motor coupling part 451a may be formed at one end of the second-1 part 451. That is, the second motor coupling part 451a may be directly coupled to the second driving pulley 450, and when the second motor coupling part 451a, which is coupled to a second jaw driving motor (not shown), rotates, the second-1 part 451, the second driving pulley rotation shaft 453, and the second-2 part 452, which are directly coupled thereto, may rotate together. That is, when the second motor coupling part 451a rotates, the second driving pulley 450 may rotate.

A groove around which a wire is wound may be formed on the side of another end of the second-1 part 451. That is, the groove may be formed in a region of the second-1 part 451 adjacent to the second-2 part 452. Also, a groove around which a wire is wound may be formed in a region of the second-2 part 452 adjacent to the second-1 part 451. One end of the second-2 part 452 may be adjacent to the second-1 part 451, and a second driving pulley head 452a may be formed at another end of the second-2 part 452.

In addition, the wire wound around the second-1 part 451 and the wire wound around the second-2 part 452 may be wound in opposite directions to each other. For example, the wire wound around the second-1 part 451 may be wound in the clockwise direction, and the wire wound around the second-2 part 452 may be wound in the counterclockwise direction. Accordingly, when the second driving pulley 450 rotates in one direction, one wire may be wound around the second driving pulley 450, and another wire may be unwound from the second driving pulley 450.

Similarly, the third driving pulley 460 may include a third-1 part 461, a third-2 part 462, and a third driving pulley rotation shaft 463. In this case, the third-1 part 461 and the third-2 part 462 are portions to which wires are respectively connected and wound, and may be coupled to the third driving pulley rotation shaft 463 to rotate together with the third driving pulley rotation shaft 463.

The third motor coupling part 461a may be formed at one end of the third-1 part 461. That is, the third motor coupling part 461a may be directly coupled to the third driving pulley 460, and when the third motor coupling part 461a, which is coupled to a pitch driving motor (not shown), rotates, the third-1 part 461, the third driving pulley rotation shaft 463, and the third-2 part 462, which are directly coupled thereto, may rotate together. That is, when the third motor coupling part 461a rotates, the third driving pulley 460 may rotate.

A groove around which a wire is wound may be formed on the side of another end of the third-1 part 461. That is, the groove may be formed in a region of the third-1 part 461 adjacent to the third-2 part 462. Also, a groove around which a wire is wound may be formed in a region of the third-2 part 462 adjacent to the third-1 part 461. One end of the third-2 part 462 may be adjacent to the third-1 part 461, and a third driving pulley head 462a may be formed at another end of the third-2 part 462.

In addition, the wire wound around the third-1 part 461 and the wire wound around the third-2 part 462 may be wound in opposite directions to each other. For example, the wire wound around the third-1 part 461 may be wound in the clockwise direction, and the wire wound around the third-2 part 462 may be wound in the counterclockwise direction. Accordingly, when the third driving pulley 460 rotates in one direction, one wire may be wound around the third driving pulley 460, and another wire may be unwound from the third driving pulley 460.

A wire 501 and a wire 503, which are first jaw wires, may be connected to the first driving pulley 440. In detail, the wire 501 may be wound around the first-2 part 442, and the wire 503 may be wound around the first-1 part 441. As described above, when the first driving pulley 440 rotates in one direction, the wire 501 and the wire 503 may each be wound around or unwound from the first driving pulley 440, thereby transmitting a driving force to the first jaw of the end tool 30.

A wire 502 and a wire 504, which are second jaw wires, may be connected to the second driving pulley 450. In detail, the wire 502 may be wound around the second-1 part 451 and the wire 504 may be wound around the second-2 part 452. As described above, when the second driving pulley 450 rotates in one direction, the wire 502 and the wire 504 may each be wound around or unwound from the second driving pulley 450, thereby transmitting a driving force to the second jaw of the end tool 30.

A wire 505 and a wire 507, which are pitch wires, may be connected to the third driving pulley 460. For example, the wire 505 may be wound around the third-1 part 461 and the wire 507 may be wound around the third-2 part 462. As described above, when the third driving pulley 460 rotates in one direction, the wire 505 and the wire 507 may each be wound around or unwound from the third driving pulley 460, thereby transmitting a driving force to a pitch pulley of the end tool 30.

In some embodiments, the fourth motor coupling part 471a may be formed at one end of the fourth driving pulley 470. That is, the fourth motor coupling part 471a may be directly coupled to the fourth driving pulley 470, and when the fourth motor coupling part 471a, which is coupled to a roll driving motor (not shown), rotates, the fourth driving pulley 470, which is directly coupled thereto, may rotate together. That is, when the fourth motor coupling part 471a rotates, the fourth driving pulley 470 may rotate.

In this case, the fourth driving pulley 470 may be coupled to a roll driving gear 472. In detail, the roll driving gear 472 has a structure with a central through-hole, and the fourth driving pulley 470 may be inserted into the through-hole of the roll driving gear 472 and coupled to the roll driving gear 472. Accordingly, the fourth driving pulley 470 and the roll driving gear 472 may rotate around a same axis.

Also, the roll driving gear 472 may be engaged with a gear 492 formed on the shaft connector 491. Accordingly, when the fourth motor coupling part 471a coupled to the roll driving motor rotates, the fourth driving pulley 470 and the roll driving gear 472 may rotate, thereby allowing the shaft connector 491 to rotate. Therefore, a roll rotational motion of the connection part 310 and the end tool 30 may be controlled.

Although the fifth driving pulley 480 illustrated in the drawings is not connected to any gear device or wire, the fifth driving pulley 480 may perform a separate function by being combined with an additional component.

In an embodiment, the first driving pulley 440 has been described as a first jaw driving pulley, the second driving pulley 450 as a second jaw driving pulley, the third driving pulley 460 as a pitch driving pulley, and the fourth driving pulley 470 as a roll driving pulley. However, the concept of the disclosure is not limited thereto, and the first driving pulley 440, the second driving pulley 450, the third driving pulley 460, the fourth driving pulley 470, and the fifth driving pulley 480 may each be formed in various positions and sizes suitable for the configuration of the driving part 40, and may perform various functions.

Hereinafter, the transition pulley assembly and the auxiliary pulley assembly of the driving part 40 according to an embodiment will be described in detail.

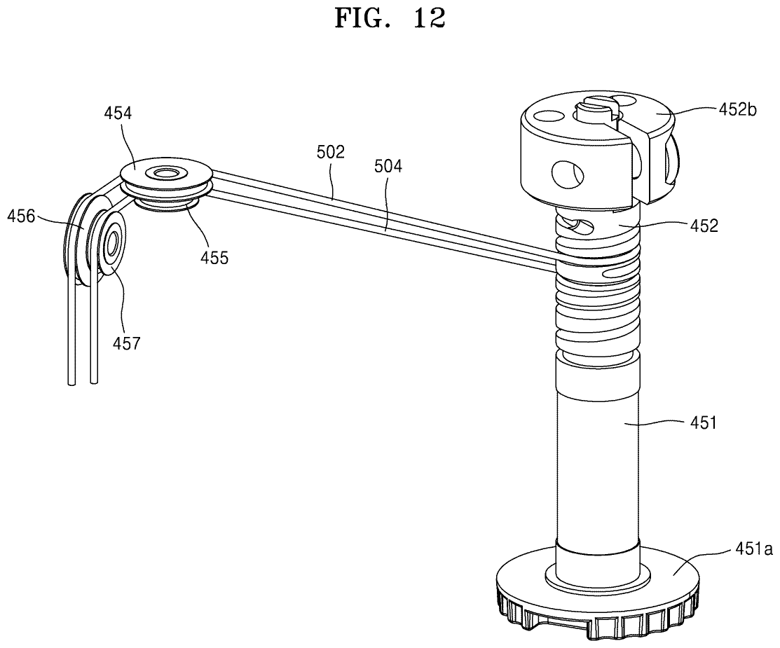

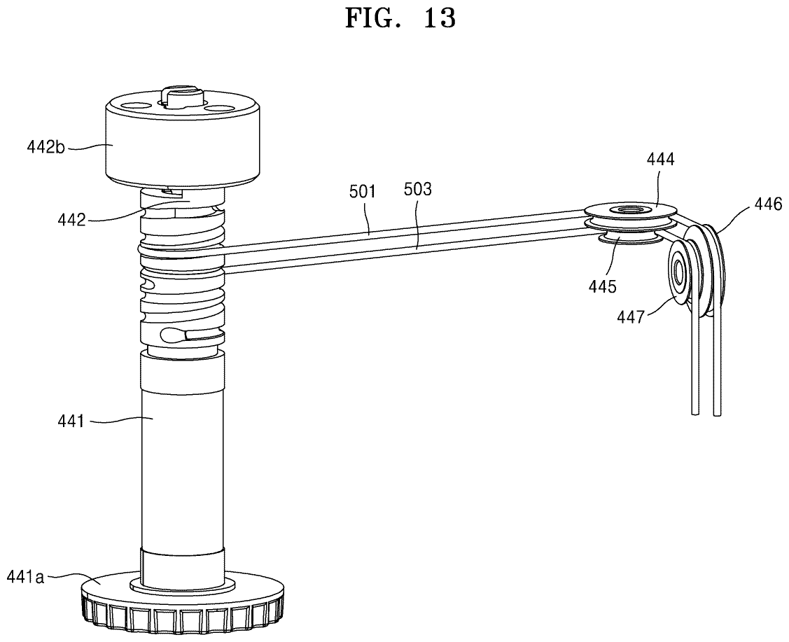

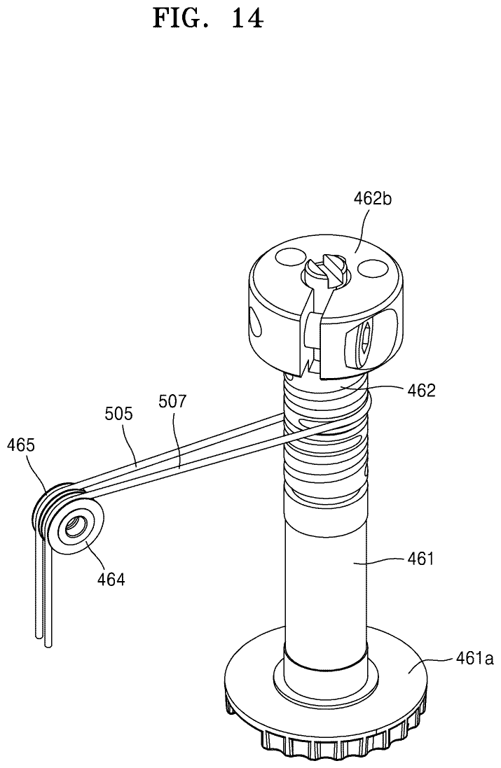

FIG. 12 is an exploded view illustrating a configuration related to a first driving pulley among the configuration of pulleys and wires of the driving part shown in FIG. 6, FIG. 13 is an exploded view illustrating a configuration related to a second driving pulley among the configuration of pulleys and wires of the driving part shown in FIG. 6, and FIG. 14 is an exploded view illustrating a configuration related to a third driving pulley among the configuration of pulleys and wires of the driving part shown in FIG. 6.

Further referring to FIGS. 12 to 14, the wire assembly of the surgical instrument according to an embodiment may include the wire 501, the wire 502, the wire 503, the wire 504, the wire 505, and the wire 507.

In this case, the wire 501 and the wire 503 may form a pair to serve as a first jaw wire. The wire 502 and the wire 504 may form a pair to serve as a second jaw wire. In this regard, components encompassing the wire 501 and the wire 503, which constitute the first jaw wire, and the wire 502 and the wire 504, which constitute the second jaw wire, may be referred to as a jaw wire. In addition, the wire 505 and the wire 507 may form a pair to serve as a pitch wire.

The driving part 40 according to an embodiment may include a pulley 444, a pulley 445, a pulley 446, a pulley 447, a pulley 454, a pulley 455, a pulley 456, a pulley 457, a pulley 464, and a pulley 465, which guide paths of wires extending from the driving pulleys.

In this case, the pulley 444, the pulley 445, the pulley 446, and the pulley 447 may be pulleys related to a rotational motion of a first jaw. Also, the pulley 454, the pulley 455, the pulley 456, and the pulley 457 may be pulleys related to a rotational motion of a second jaw. In addition, the pulley 464 and the pulley 465 may be pulleys related to a pitch rotation of the end tool 30. However, the concept of the disclosure is not limited thereto.

In detail, the pulley 446 and the pulley 447 function as transition pulleys of the first jaw wire, and these components may be collectively referred to as a first group of transition pulleys. Also, the pulley 456 and the pulley 457 function as transition pulleys of the second jaw wire, and these components may be collectively referred to as a second group of transition pulleys. Moreover, the pulley 464 and the pulley 465 function as transition pulleys of the pitch wire, and these components may be collectively referred to as a third group of transition pulleys.

As described above, the transition pulley assembly may include the first group of transition pulleys, the second group of transition pulleys, and the third group of transition pulleys.

In this case, the first group of transition pulleys, the second group of transition pulleys, and the third group of transition pulleys may guide wire assemblies entering from different directions. This will be described in detail below.

In some embodiments, the pulley 444 and the pulley 445 function as auxiliary pulleys of the first jaw wire, and these components may be collectively referred to as a first group of auxiliary pulleys. Also, the pulley 454 and the pulley 455 function as auxiliary pulleys of the second jaw wire, and these components may be collectively referred to as a second group of auxiliary pulleys.

Furthermore, the driving part 40 according to an embodiment may include a rotation shaft AX1, a rotation shaft AX2, a rotation shaft AX3, a rotation shaft AX4, and a rotation shaft AX5. In this case, the rotation shaft AX1 may function as a rotation shaft of the first group of transition pulleys, the rotation shaft AX3 may function as a rotation shaft of the second group of transition pulleys, and the rotation shaft AX5 may function as a rotation shaft of the third group of transition pulleys. In addition, the rotation shaft AX2 may function as a rotation shaft of the first group of auxiliary pulleys, and the rotation shaft AX4 may function as a rotation shaft of the second group of auxiliary pulleys. One or more pulleys may be mounted on each of these rotation shafts, and this will be described in detail below.

Hereinafter, each of the components will be described in further detail.

The pulley 446, the pulley 447, the pulley 456, the pulley 457, the pulley 464, and the pulley 465 may be positioned in the wire guide assembly 430 to be described below. In detail, the pulley 446 and the pulley 447 may be coupled to the rotation shaft AX2 and formed to be rotatable around the rotation shaft AX2. In addition, the pulley 456 and the pulley 457 may be coupled to the rotation shaft AX4 and formed to be rotatable around the rotation shaft AX4. Moreover, the pulley 464 and the pulley 465 may be coupled to the rotation shaft AX5 and formed to be rotatable around the rotation shaft AX5.

In this case, the rotation shaft AX2, the rotation shaft AX4, and the rotation shaft AX5 may be positioned on a same plane. In other words, the rotation shaft of the first group of transition pulleys, the rotation shaft of the second group of transition pulleys, and the rotation shaft of the third group of transition pulleys may be positioned on a same plane.

In detail, the rotation shaft AX2 and the rotation shaft AX4 may be positioned parallel to each other, and the rotation shaft AX5 may be positioned perpendicular to the rotation shaft AX2 and the rotation shaft AX4.

That is, the rotation shaft of the first group of transition pulleys and the rotation shaft of the second group of transition pulleys may be positioned parallel to each other. The rotation shaft of the third group of transition pulleys may be positioned perpendicular to the rotation shaft of the first group of transition pulleys and the rotation shaft of the second group of transition pulleys.

In this case, the transition pulley assemblies may be formed as a pair to allow two strands of wire wound around the driving pulley assembly to be wound around the transition pulley assemblies, respectively. For example, the pulley 446 may function as a first transition pulley, and the pulley 447 may function as a second transition pulley. In addition, the pulley 456 may function as a first transition pulley, and the pulley 457 may function as a second transition pulley. Also, the pulley 464 may function as a first transition pulley, and the pulley 465 may function as a second transition pulley.

In this case, a diameter of the first transition pulley may be different from a diameter of the second transition pulley. For example, a diameter of the pulley 446 may be different from a diameter of the pulley 447, and a diameter of the pulley 456 may be different from a diameter of the pulley 457. In detail, the diameter of the pulley 446 may be greater than the diameter of the pulley 447. Similarly, the diameter of the pulley 456 may be greater than the diameter of the pulley 457.

In another embodiment, a diameter of the first transition pulley may be equal to a diameter of the second transition pulley. For example, a diameter of the pulley 464 may be equal to a diameter of the pulley 465.

In other words, the first group of transition pulleys includes a pair of transition pulleys having different diameters, the second group of transition pulleys also includes a pair of transition pulleys having different diameters, and the third group of transition pulleys may include a pair of transition pulleys having an equal diameter.

In an embodiment, a diameter of the first transition pulley of the first group of transition pulleys may be equal to a diameter of the first transition pulley of the second group of transition pulleys. Also, a diameter of the second transition pulley of the first group of transition pulleys may be equal to a diameter of the second transition pulley of the second group of transition pulleys.

In addition, diameters of the first transition pulley and the second transition pulley of the third group of transition pulleys may be equal to the diameter of the second transition pulley of the first group of transition pulleys and equal to the diameter of the second transition pulley of the second group of transition pulleys. For example, diameters of the pulley 446, the pulley 447, the pulley 456, the pulley 457, the pulley 464, and the pulley 465 may all be equal. However, the concept of the disclosure is not limited thereto.

The auxiliary pulley assembly may be positioned adjacent to the transition pulley assembly. That is, the pulley 444, the pulley 445, the pulley 454, and the pulley 455 may be positioned adjacent to the wire guide assembly 430. In detail, the first group of auxiliary pulleys may be positioned adjacent to the first group of transition pulleys, and the second group of auxiliary pulleys may be positioned adjacent to the second group of transition pulleys.

For example, the pulley 444 and the pulley 445 may be coupled to the rotation shaft AX1 and formed to be rotatable around the rotation shaft AX1. In addition, the pulley 454 and the pulley 455 may be coupled to the rotation shaft AX3 and formed to be rotatable around the rotation shaft AX3.

In this case, the rotation shaft AX1 and the rotation shaft AX3 may be positioned parallel to each other. Also, the rotation shaft AX1 and the rotation shaft AX3 may be positioned parallel to a rotation shaft of the driving pulley assembly.

The auxiliary pulley assemblies may be formed as a pair to allow two strands of wire wound around the driving pulley assembly to be wound around the auxiliary pulley assemblies, respectively. For example, the pulley 444 may function as a first auxiliary pulley, and the pulley 445 may function as a second auxiliary pulley. In addition, the pulley 454 may function as a first auxiliary pulley, and the pulley 455 may function as a second auxiliary pulley.

In this case, a diameter of the first auxiliary pulley may be different from a diameter of the second auxiliary pulley. For example, a diameter of the pulley 444 may be different from a diameter of the pulley 445, and a diameter of the pulley 454 may be different from a diameter of the pulley 455. In detail, the diameter of the pulley 444 may be greater than the diameter of the pulley 445. Similarly, the diameter of the pulley 454 may be greater than the diameter of the pulley 455.

In other words, the first group of auxiliary pulleys may include a pair of auxiliary pulleys having different diameters, and the second group of auxiliary pulleys may include a pair of auxiliary pulleys having different diameters.

In an embodiment, a diameter of the first auxiliary pulley of the first group of auxiliary pulleys may be equal to a diameter of the first auxiliary pulley of the second group of auxiliary pulleys. Also, a diameter of the second auxiliary pulley of the first group of auxiliary pulleys may be equal to a diameter of the second auxiliary pulley of the second group of auxiliary pulleys. For example, the diameter of the pulley 444 may be equal to the diameter of the pulley 445, and the diameter of the pulley 454 may be equal to the diameter of the pulley 455. However, the concept of the disclosure is not limited thereto.

The driving part 40 according to an embodiment includes the transition pulley assembly and the auxiliary pulley assembly as described above, and may guide a path of the wire assembly.

The transition pulley assembly is positioned such that at least a portion of the wire assembly is in contact therewith, and may guide the wire assembly extending from the driving pulley assembly to the inside of the connection part 310.

In detail, the transition pulley assembly may guide the wire assembly into the shaft connector 491. Accordingly, the transition pulley assembly may be positioned adjacent to one end of the shaft connector 491.

The auxiliary pulley assembly is positioned adjacent to the transition pulley assembly, and may change a path of the wire assembly between the transition pulley assembly and the driving pulley assembly.

That is, the path of the wire assembly extending from the driving pulley assembly may be first changed by the auxiliary pulley assembly and then changed again by the transition pulley assembly.

In detail, the wire 501, which is the first jaw wire, may extend from the first driving pulley 440 and be wound in sequence to be in contact with at least a portion of the pulley 444 and the pulley 446, and then be connected to the end tool 30 through the connection part 310.

In other words, the wire 501 having one end wound around the first driving pulley 440 may pass through the first driving pulley 440, the first auxiliary pulley 444, and the first transition pulley 446 in sequence, and then be connected to the end tool 30 through the connection part 310.

From another perspective, the wire 501, which is the first jaw wire, may pass through the end tool 30 and the connection part 310 and enter the driving part 40, then be wound around the pulley 446 and the pulley 444 in sequence, and then be fixedly coupled to the first driving pulley 440.

In addition, the wire 503, which is the first jaw wire, may extend from the first driving pulley 440 and be wound in sequence to be in contact with at least a portion of the pulley 445 and the pulley 447, and then be connected to the end tool 30 through the connection part 310.

In some embodiments, the wire 502, which is the second jaw wire, may extend from the second driving pulley 450 and be wound in sequence to be in contact with at least a portion of the pulley 454 and the pulley 456, and then be connected to the end tool 30 through the connection part 310.

Also, the wire 504, which is the second jaw wire, may extend from the second driving pulley 450 and be wound in sequence to be in contact with at least a portion of the pulley 455 and the pulley 457, and then be connected to the end tool 30 through the connection part 310.

In some embodiments, the wire 505 may extend from the third driving pulley 460 and be wound in sequence to be in contact with at least a portion of the pulley 465, and then be connected to the end tool 30 through the connection part 310, and the wire 507 may extend from the third driving pulley 460 and be wound in sequence to be in contact with at least a portion of the pulley 464, and then be connected to the end tool 30 through the connection part 310.

As described above, wires entering from different directions may be guided into the shaft connector 491 by respective transition pulleys.

The wire assembly extending from the driving pulley assembly may enter the auxiliary pulley assembly in a first direction, pass through the auxiliary pulley assembly, and be redirected toward a second direction. Then, the wire assembly may pass through the transition pulley assembly and be redirected toward a third direction. In this case, the third direction may not be parallel to the first direction.

Hereinafter, an arrangement relationship between auxiliary pulleys and transition pulleys will be described in detail.

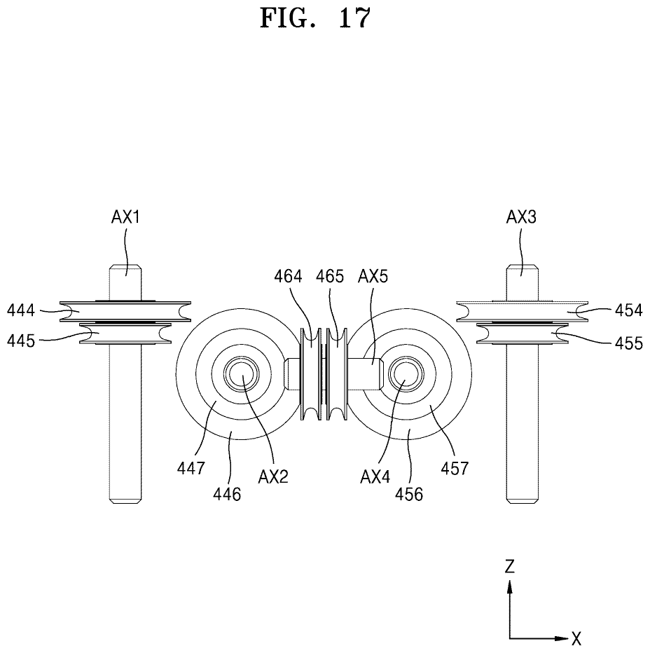

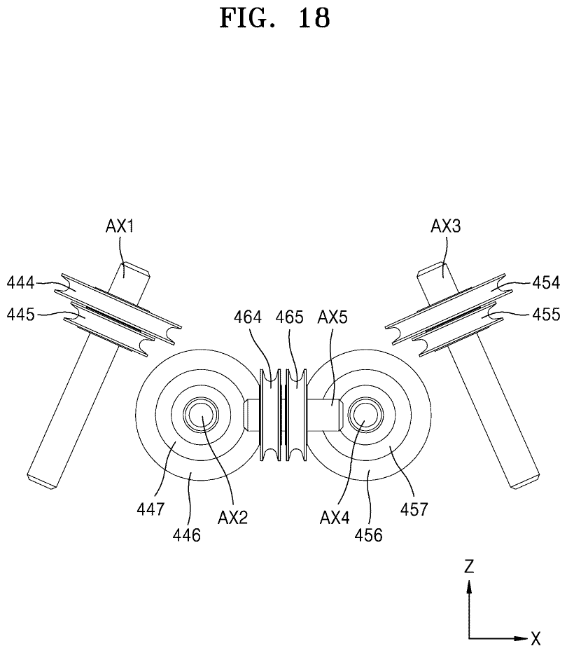

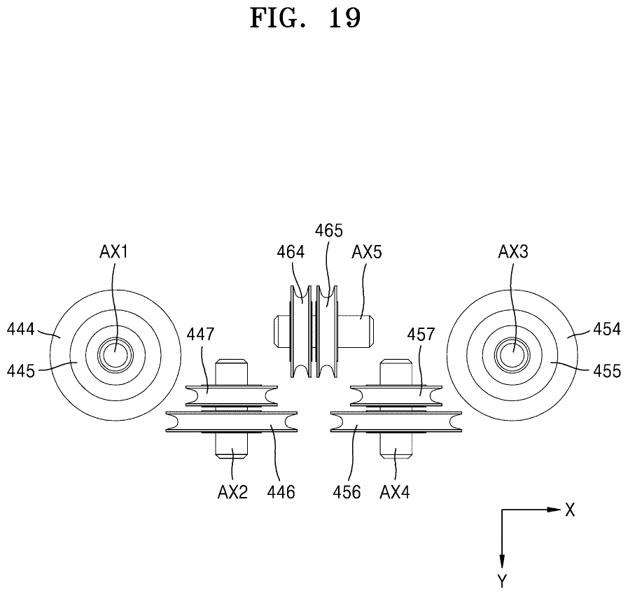

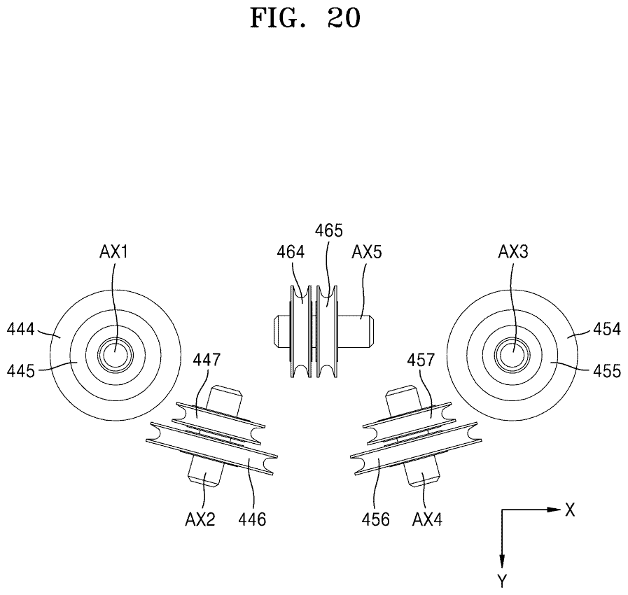

FIG. 17 is a diagram for explaining an arrangement state of transition pulleys and auxiliary pulleys shown in FIG. 7. FIG. 18 is a diagram for explaining a modified example of the arrangement state of the auxiliary pulleys of FIG. 17. FIG. 19 is a diagram of the transition pulleys and the auxiliary pulleys shown in FIG. 17, taken from a different angle. FIG. 20 is a diagram for explaining a modified example of an arrangement state of the transition pulleys of FIG. 19.

Referring to FIGS. 17 to 20, the auxiliary pulley assembly according to an embodiment may be positioned so as not to be parallel to the transition pulley assembly.

In detail, the first group of auxiliary pulleys may be positioned so as not to be parallel to the first group of transition pulleys. The first auxiliary pulley 444 may be positioned so as not to be parallel to the first transition pulley 446, and the second auxiliary pulley 445 may be positioned so as not to be parallel to the second transition pulley 447.

Also, the second group of auxiliary pulleys may be positioned so as not to be parallel to the second group of transition pulleys. The first auxiliary pulley 454 may be positioned so as not to be parallel to the first transition pulley 456, and the second auxiliary pulley 455 may be positioned so as not to be parallel to the second transition pulley 457.

In other words, a plane including a rotational surface of the transition pulley assembly and a plane including a rotational surface of the auxiliary pulley assembly may be perpendicular to each other.

For example, a plane including a rotational surface of the first transition pulley 446 and a plane including a rotational surface of the first auxiliary pulley 444 may be perpendicular to each other. Also, a plane including a rotational surface of the first transition pulley 456 and a plane including a rotational surface of the first auxiliary pulley 454 may be perpendicular to each other.

The rotation shaft AX1 of the first group of auxiliary pulleys and the rotation shaft AX2 of the first group of transition pulleys may not be parallel to each other. In addition, the rotation shaft AX3 of the second group of auxiliary pulleys and the rotation shaft AX4 of the second group of transition pulleys may not be parallel to each other.

In other words, the auxiliary pulley assembly may be formed to be rotatable around a first axis, and the transition pulley assembly may be formed to be rotatable around a second axis that is not parallel to the first axis.

Referring again to FIGS. 17 and 19, in an embodiment, the rotation shaft AX1 of the first group of auxiliary pulleys and the rotation shaft AX3 of the second group of auxiliary pulleys may be positioned parallel to each other. Also, the rotation shaft AX2 of the first group of transition pulleys and the rotation shaft AX4 of the second group of transition pulleys may be positioned parallel to each other.

Referring to FIG. 18, in an embodiment, the rotation shaft AX1 of the first group of auxiliary pulleys and the rotation shaft AX3 of the second group of auxiliary pulleys may be positioned so as not to be parallel to each other. In other words, an extension line of the rotation shaft AX1 of the first group of auxiliary pulleys and an extension line of the rotation shaft AX3 of the second group of auxiliary pulleys may be positioned to cross each other.

Referring to FIG. 20, in an embodiment, the rotation shaft AX2 of the first group of transition pulleys and the rotation shaft AX4 of the second group of transition pulleys may be positioned so as not to be parallel to each other. In other words, an extension line of the rotation shaft AX2 of the first group of transition pulleys and an extension line of the rotation shaft AX4 of the second group of transition pulleys may be positioned to cross each other.

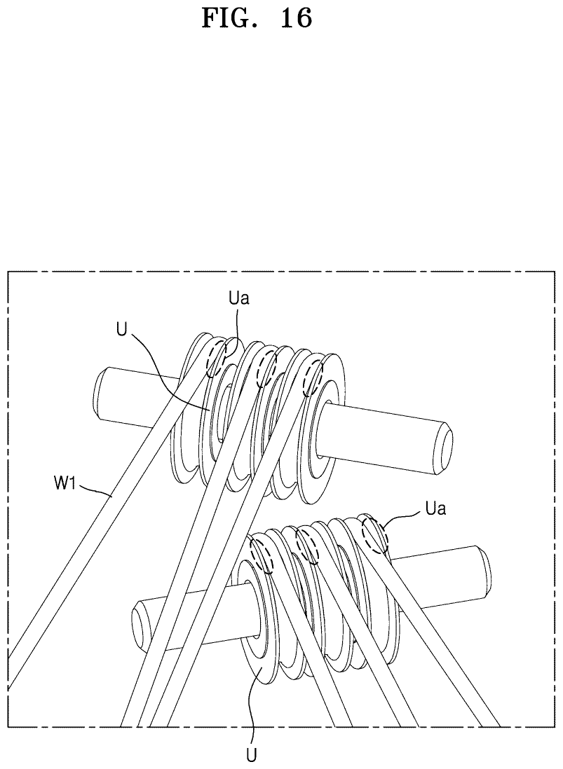

FIG. 16 is an exploded view illustrating a configuration of pulleys and wires according to a comparative example.

As shown in FIG. 16, wires may be respectively wound around a plurality of pulleys. In this case, when the plurality of pulleys are coupled to one rotation shaft, the plurality of pulleys are constrained to be positioned in parallel.

Also, travel paths of the wires wound around the respective pulleys may be different from each other, and the travel paths of the wires may be bent at a certain angle on rotational surfaces of the pulleys.

Accordingly, as in the comparative example, a wire W1 wound around a pulley U may be unintentionally in contact with a side portion Ua of the pulley U.

When a contact surface between a pulley and a wire increases as described above, friction between the wire and the pulley may increase. In this case, there are problems that the life of the wire is reduced and the efficiency of power transmission through the wire deteriorates. Therefore, a contact area between the pulley and the wire needs to be minimized to minimize power loss by reducing friction between the pulley and the wire.

In contrast, the surgical instrument according to an embodiment may minimize a friction surface between the pulley and the wire by applying a structure that changes a path of the wire in the driving part 40 twice.

Therefore, the surgical instrument according to an embodiment may improve the life of the wire and the pulley, and may improve power transmission efficiency of the wire.

The driving part 40 according to an embodiment of may prevent unnecessary contact from occurring between the wire and the pulley by optimizing the arrangement of the transition pulley assembly and the auxiliary pulley assembly.

Referring to FIG. 12, a path of the wire 502 may include a first straight path passing between the first auxiliary pulley 454 and the first transition pulley 456.

The first straight path may be referred to as a path corresponding to a straight distance connecting from a point where the wire 502 exits from the first auxiliary pulley 454 to a point where the wire 502 enters the first transition pulley 456.

In this case, a groove on which the wire 502 is wound around the first auxiliary pulley 454 and a groove on which the wire 502 is wound around the first transition pulley 456 may be positioned parallel to the first straight path.

Similarly, a path of the wire 504 may include a second straight path passing between the second auxiliary pulley 455 and the second transition pulley 457.

The second straight path may be referred to as a path corresponding to a straight distance connecting from a point where the wire 504 exits from the second auxiliary pulley 455 to a point where the wire 504 enters the second transition pulley 457.

In this case, a groove on which the wire 504 is wound around the second auxiliary pulley 455 and a groove on which the wire 504 is wound around the second transition pulley 457 may be positioned parallel to the second straight path.

In other words, a path of the wire assembly includes a straight path passing between the auxiliary pulley assembly and the transition pulley assembly, and a groove in which the wire assembly is wound around the auxiliary pulley assembly and a groove in which the wire assembly is wound around the transition pulley assembly may be positioned parallel to a straight path.

As described above, according to an embodiment, contact of the wire with a side portion of the pulley may be minimized, and friction may be prevented from increasing by preventing unnecessary contact between the wire and the pulley.

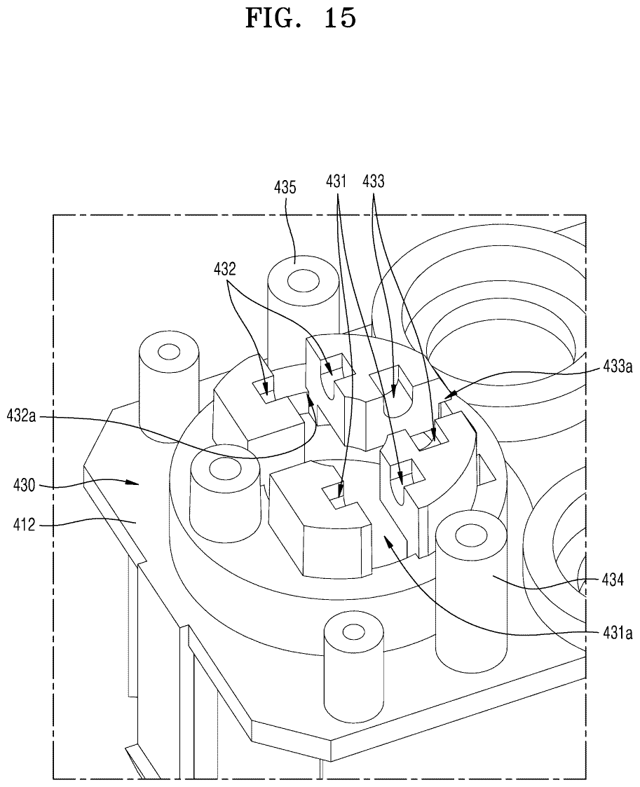

FIG. 15 is a perspective view of the wire guide assembly 430 of the driving part 40 of FIG. 6.

The wire guide assembly 430 may be positioned on the plate part 410. As described above, the wire guide assembly 430 may be a portion in which the transition pulley assembly is positioned.

The wire guide assembly 430 may include a pulley accommodation space in which the transition pulley assembly is positioned, and a pulley shaft accommodation space in which a rotation shaft of the transition pulley assembly is accommodated.

In detail, the wire guide assembly 430 may include a first pulley accommodation space 431a in which a first group of transition pulleys is positioned, a second pulley accommodation space 432a in which a second group of transition pulleys is positioned, and a third pulley accommodation space 433a in which a third group of transition pulleys is positioned.

In other words, the pulley 446 and the pulley 447 may be accommodated in the first pulley accommodation space 431a, the pulley 456 and the pulley 457 may be accommodated in the second pulley accommodation space 432a, and the pulley 464 and the pulley 465 may be accommodated in the third pulley accommodation space 433a. Each pulley accommodation space may accommodate at least some transition pulleys. In this case, the respective pulley accommodation spaces may be connected to one another.

In addition, the wire guide assembly 430 may include a first pulley shaft accommodation space 431 in which rotation shaft AX2 is accommodated, a second pulley shaft accommodation space 432 in which rotation shaft AX4 is accommodated, and a third pulley shaft accommodation space 433 in which rotation shaft AX5 is accommodated.

In this regard, the first pulley shaft accommodation space 431, the second pulley shaft accommodation space 432, and the third pulley shaft accommodation space 433 may be positioned on a same plane.

In detail, the first pulley shaft accommodation space 431 and the second pulley shaft accommodation space 432 may be positioned parallel to each other, and the third pulley shaft accommodation space 433 may be positioned perpendicular to the first pulley shaft accommodation space 431 and the second pulley shaft accommodation space 432.

Hereinafter, a driving part 60 according to another embodiment will be described in further detail.

The driving part 60 according to another embodiment has a characteristically different configuration of transition pulleys and auxiliary pulleys compared to the embodiment described with reference to FIG. 6 or the like. Hereinafter, the configuration that differs from the aforementioned embodiment will be described.

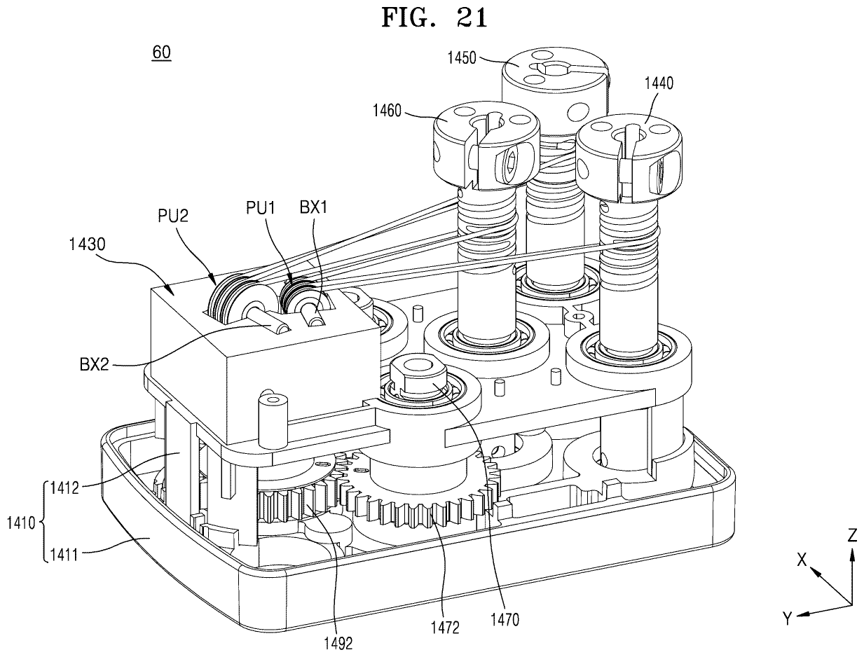

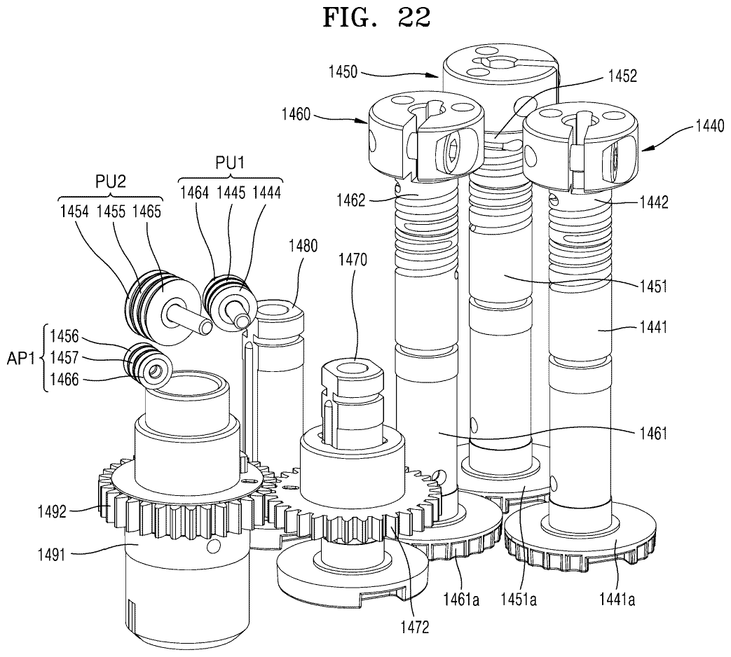

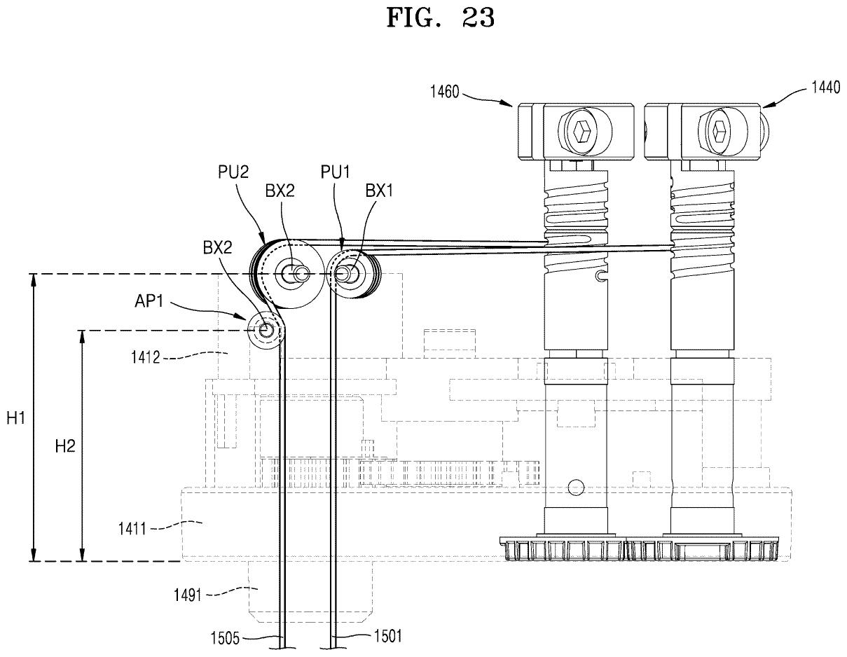

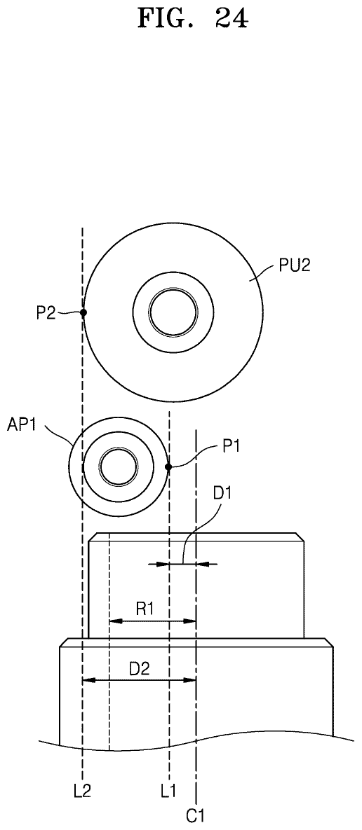

FIG. 21 is a perspective view of the driving part 60 according to another embodiment, FIG. 22 is an exploded view illustrating a configuration of pulleys of the driving part 60 shown in FIG. 21, and FIG. 23 is a side view of the driving part 60 of FIG. 21. FIG. 24 is a diagram for explaining an arrangement state of transition pulleys and auxiliary pulleys of the driving part 60 of FIG. 21.

Referring to FIGS. 21 to 24, the driving part 60 according to another embodiment may include a plate part 1410, a cover (not shown), a wire guide assembly 1430, a driving pulley assembly (reference numeral not shown), a transition pulley assembly (reference numeral not shown), an auxiliary pulley assembly (reference numeral not shown), a wire assembly (reference numeral not shown), and a shaft connector 1491.

The plate part 1410, the cover (not shown), the driving pulley assembly (reference numeral not shown), and the shaft connector 1491 of the driving part 60 according to another embodiment are substantially the same within a corresponding range as the plate part 410, the cover (not shown), the driving pulley assembly (reference numeral not shown), and the shaft connector 491 of the driving part 40 according to the embodiment described with reference to FIG. 6 or the like, and thus, a detailed description thereof will be omitted herein.

Also, the driving pulley assembly may include a first driving pulley 1440, a second driving pulley 1450, a third driving pulley 1460, a fourth driving pulley 1470, and a fifth driving pulley 1480.

The first driving pulley 1440, the second driving pulley 1450, the third driving pulley 1460, the fourth driving pulley 1470, and the fifth driving pulley 1480 of the driving part 60 according to another embodiment are substantially the same within a corresponding range as the first driving pulley 440, the second driving pulley 450, the third driving pulley 460, the fourth driving pulley 470, and the fifth driving pulley 480 of the driving part 40 according to the embodiment described with reference to FIG. 6 or the like, and thus, a detailed description will be omitted herein.

The driving part 60 according to another embodiment may include a pulley 1444, a pulley 1445, a pulley 1454, a pulley 1455, a pulley 1456, a pulley 1457, a pulley 1464, a pulley 1465, and a pulley 1466, which guide paths of wires extending from the driving pulleys.

Also, the driving part 60 according to another embodiment may include a rotation shaft BX1, a rotation shaft BX2, and a rotation shaft BX3.

In this case, the pulley 1444, the pulley 1445, and the pulley 1464 may be coupled to the rotation shaft BX1 and formed to be rotatable around the rotation shaft BX1. Also, the pulley 1454, the pulley 1455, and the pulley 1465 may be formed to be rotatable around the rotation shaft BX2. In addition, the pulley 1456, the pulley 1457, and the pulley 1466 may be formed to be rotatable around the rotation shaft BX3.

In this regard, the pulley 1444, the pulley 1445, and the pulley 1464, which are shaft-coupled to the rotation shaft BX1, function as first transition pulleys, and these components may be collectively referred to as a first group of transition pulleys PU1. Also, the pulley 1454, the pulley 1455, and the pulley 1465, which are shaft-coupled to the rotation shaft BX2, function as second transition pulleys, and these components may be collectively referred to as a second group of transition pulleys PU2.

In other words, the transition pulley assembly may include the first group of transition pulleys PU1 and the second group of transition pulleys PU2. In other words, the transition pulley assembly may include a plurality of transition pulleys positioned to be rotatable around a same shaft.

The transition pulley assembly is positioned such that at least a portion of the wire assembly is in contact therewith, and may guide the wire assembly extending from the driving pulley assembly to the inside of the connection part 310.

For example, among the first group of transition pulleys PU1, the pulley 1444 and the pulley 1445 may guide a path of the first jaw wire extending from the first driving pulley 1440, and among the second group of transition pulleys PU2, the pulley 1454 and the pulley 1455 may guide a path of the second jaw wire extending from the second driving pulley 1450. Also, the pulley 1464 and the pulley 1465 may guide a path of a pitch wire extending from the third driving pulley 1460. However, the concept of the disclosure is not limited thereto.

In some embodiments, the pulley 1456, the pulley 1457, and the pulley 1466, which are shaft-coupled to the rotation shaft BX3, function as auxiliary pulleys and may be referred to as a first group of auxiliary pulleys AP1.

In other words, the auxiliary pulley assembly may include the first group of auxiliary pulleys AP1. From another perspective, the auxiliary pulley assembly may include a plurality of auxiliary pulleys positioned to be rotatable around a same shaft.

The auxiliary pulley assembly is positioned adjacent to the transition pulley assembly and may change a path of the wire assembly between the transition pulley assembly and the connection part 310.

In detail, the first group of auxiliary pulleys AP1 may be positioned adjacent to the second group of transition pulleys PU2. In this case, the first group of auxiliary pulleys AP1 may be positioned closer to the shaft connector 491 than the second group of transition pulleys PU2. In other words, a distance from one surface of the plate part 410 to the auxiliary pulley assembly may be shorter than a distance to the transition pulley assembly.

From another perspective, when a distance from a coupling surface of the base plate 411 to the rotation shaft BX3, which is a rotation shaft of the first group of auxiliary pulleys AP1, is represented as H2, and a distance from the coupling surface of the base plate 411 to the rotation shaft BX2, which is a rotation shaft of the second group of transition pulleys PU2, is represented as H1, H2 may be less than H1.

FIG. 24 is a schematic diagram of the shaft connector 491, the first group of auxiliary pulleys AP1, and the second group of transition pulleys PU2. Illustration of the wire is omitted in this drawing.

Referring to FIG. 24, a vertical distance between an exit point P2, which is a point where the wire exits from the transition pulley, and a central axis of the shaft connector 491 may be represented as D2, a vertical distance between an entry point P1, which is a point where the wire enters the auxiliary pulley, and a central axis C1 of the shaft connector 491 may be represented as D1, and an inner radius of the shaft connector 491 may be represented as R1.

In this case, D1 may be less than D2. In other words, the entry point P1 of the auxiliary pulley may be positioned closer to the central axis C1 of the connection part 310 than the exit point P2 of the transition pulley. From another perspective, the exit point P2 of the transition pulley may be positioned farther from the central axis C1 of the connection part 310 than the entry point P1 of the auxiliary pulley.

Also, D1 may be less than R1. In other words, a distance between the exit point P2 of the auxiliary pulley and the central axis C1 of the connection part 310 may be shorter than an inner radius length of the connection part 310.

By positioning the pulleys as described above, a wire passing through the transition pulley may be positioned inside the connection part 310 without interference.

Hereinafter, a detailed description will be provided based on each component.

The pulley 1456, the pulley 1457, and the pulley 1466 may be positioned adjacent to the pulley 1454, the pulley 1455, and the pulley 1465, respectively.

In detail, the pulley 1456 is positioned such that a wire passing through the pulley 1454 is in contact with at least a portion thereof, and may change a path of the wire. Also, the pulley 1457 is positioned such that a wire passing through the pulley 1455 is in contact with at least a portion thereof, and may change a path of the wire. Also, the pulley 1466 is positioned such that a wire passing through the pulley 1465 is in contact with at least a portion thereof, and may change a path of the wire.

For example, a wire 1501, which is the first jaw wire, may extend from the first driving pulley 1440 and be wound to be in contact with at least a portion of the pulley 1444, and then be connected to the end tool 30 through the connection part 310.

Also, a wire 1505, which is a pitch wire, may extend from the third driving pulley 1460 and be wound in sequence to be in contact with at least a portion of the pulley 1465 and the pulley 1466, and then be connected to the end tool 30 through the connection part 310.

In other words, the wire 1505 may pass through the pulley 1465, which is the second transition pulley, and be partially in contact with the pulley 1466, which is the auxiliary pulley, thereby being positioned close to the wire 1501. That is, a distance between the wire 1505 and the wire 1501 positioned in the shaft connector 491 may be reduced. From another perspective, a distance between the wire 1505 and the wire 1501 positioned in the connection part 310 may be reduced. Accordingly, an inner diameter of the connection part 310 may be reduced.

Therefore, the surgical instrument according to an embodiment may reduce a width of the connection part 310. Also, the surgical instrument according to an embodiment may improve the life of the wire and the pulley due to reduced friction of the wire.

According to the disclosure, a surgical instrument may be provided, wherein a path of a wire is optimized to minimize power loss of the wire.

The disclosure has been described above with a focus on the embodiments. It will be understood by those of ordinary skill in the art that various changes in form and details may be made therein without departing from the essential features of the disclosure. Therefore, the disclosed embodiments should be considered in descriptive sense only and not for purposes of limitation. The scope of the disclosure is defined not by the detailed description of the disclosure but by the appended claims, and all differences within the scope will be construed as being included in the disclosure.

What is claimed is:

1. A surgical instrument mountable on a surgical robot, the surgical instrument comprising:
an end tool including one or more jaws and having at least one degree of rotational freedom;
a wire assembly having a first portion connected to the end tool;
a connection part configured to extend in one direction, having an inside through which the wire assembly passes, and having a first side to which the end tool is coupled; and
a driving part coupled to a second side of the connection part and configured to control a rotational motion of the end tool,
wherein the driving part includes:
a driving pulley assembly configured to be rotatable around an axis and connected to a second portion of the wire assembly;
a transition pulley assembly positioned such that at least a portion of the wire assembly is in contact therewith, and configured to guide the wire assembly to the inside of the connection part, the wire assembly extending from the driving pulley assembly; and
an auxiliary pulley assembly positioned adjacent to the transition pulley assembly and configured to change a path of the wire assembly between the transition pulley assembly and the driving pulley assembly.

2. The surgical instrument of claim 1, wherein
a rotation shaft of the auxiliary pulley assembly is positioned so as not to be parallel to a rotation shaft of the transition pulley assembly.

3. The surgical instrument of claim 2, wherein
a plane including a rotational surface of the transition pulley assembly and a plane including a rotational surface of the auxiliary pulley assembly are perpendicular to each other.

4. The surgical instrument of claim 2, wherein
the rotation shaft of the auxiliary pulley assembly is positioned parallel to a rotation shaft of the driving pulley assembly.

5. The surgical instrument of claim 4, wherein
the transition pulley assembly includes a first group of transition pulleys, a second group of transition pulleys, and a third group of transition pulleys, wherein the first group of transition pulleys, the second group of transition pulleys, and the third group of transition pulleys are each configured to guide the wire assembly entering from a different direction.

6. The surgical instrument of claim 5, wherein
a rotation shaft of the first group of transition pulleys, a rotation shaft of the second group of transition pulleys, and a rotation shaft of the third group of transition pulleys are positioned on a same plane.

7. The surgical instrument of claim 6, wherein
the rotation shaft of the first group of transition pulleys and the rotation shaft of the second group of transition pulleys are positioned parallel to each other, and the rotation shaft of the third group of transition pulleys is positioned perpendicular to the rotation shaft of the first group of transition pulleys and the rotation shaft of the second group of transition pulleys.

8. The surgical instrument of claim 5, wherein
the auxiliary pulley assembly includes:
a first group of auxiliary pulleys positioned adjacent to the first group of transition pulleys; and
a second group of auxiliary pulleys positioned adjacent to the second group of transition pulleys.

9. The surgical instrument of claim 1, wherein
the transition pulley assembly includes a first transition pulley and a second transition pulley positioned to be rotatable around a same shaft, and
the auxiliary pulley assembly includes a first auxiliary pulley and a second auxiliary pulley positioned to be rotatable around a same shaft.

10. The surgical instrument of claim 9, wherein
a diameter of the first transition pulley and a diameter of the second transition pulley are different from each other, and a diameter of the first auxiliary pulley and a diameter of the second auxiliary pulley are different from each other.

11. The surgical instrument of claim 9, wherein
a diameter of the first transition pulley and a diameter of the first auxiliary pulley are equal to each other, and a diameter of the second transition pulley and a diameter of the second auxiliary pulley are equal to each other.

12. The surgical instrument of claim 1, wherein
the auxiliary pulley assembly is formed to be rotatable around a first shaft, and
the transition pulley assembly is formed to be rotatable around a second shaft that is not parallel to the first shaft.

13. The surgical instrument of claim 12, wherein
the wire assembly extending from the driving pulley assembly enters the auxiliary pulley assembly in a first direction, passes through the auxiliary pulley assembly, and is redirected toward a second direction, then enters the transition pulley assembly in the second direction, passes through the transition pulley assembly, and is redirected toward a third direction that is not parallel to the first direction.

14. The surgical instrument of claim 1, wherein the wire assembly includes two strands of wire, and
each of the transition pulley assembly and the auxiliary pulley assembly is provided as a pair to respectively guide the two strands of wire extending from the driving pulley assembly.

15. The surgical instrument of claim 1, wherein
the path of the wire assembly includes a straight path passing between the auxiliary pulley assembly and the transition pulley assembly, and
a groove in which the wire assembly is wound around the auxiliary pulley assembly and a groove in which the wire assembly is wound around the transition pulley assembly are positioned parallel to the straight path.

16. The surgical instrument of claim 1, further comprising:
a plate part on which the driving pulley assembly is positioned; and
a wire guide assembly positioned on the plate part and on which the transition pulley assembly is positioned.

17. A surgical instrument mountable on a surgical robot, the surgical instrument comprising:
an end tool including one or more jaws and having at least one degree of rotational freedom;
a wire assembly having a first portion connected to the end tool;
a connection part configured to extend in one direction, having an inside through which the wire assembly passes, and having a first side to which the end tool is coupled; and
a driving part coupled to a second side of the connection part and configured to control a rotational motion of the end tool,
wherein the driving part includes:
a driving pulley assembly configured to be rotatable around an axis and connected to a second portion of the wire assembly;
a transition pulley assembly positioned such that at least a portion of the wire assembly is in contact therewith, and configured to guide the wire assembly to the inside of the connection part, the wire assembly extending from the driving pulley assembly; and
an auxiliary pulley assembly positioned adjacent to the transition pulley assembly and configured to change a path of the wire assembly between the transition pulley assembly and the connection part.

18. The surgical instrument of claim 17, further comprising
a plate part on which the driving pulley assembly is positioned,
wherein a distance from one surface of the plate part to the auxiliary pulley assembly is shorter than a distance to the transition pulley assembly.

19. The surgical instrument of claim 17, wherein
an exit point of the transition pulley assembly is positioned farther from a central axis of the connection part than an entry point of the auxiliary pulley assembly.

20. The surgical instrument of claim 17, wherein
a distance from an exit point of the auxiliary pulley assembly to a central axis of the connection part is shorter than an inner radius length of the connection part.
